(19) United States
(12) Patent Application Publication
(10) Pub. No.: US 2008/0182808 A1
(43) Pub. Date: Jul. 31, 2008

(54) OLIGORIBONUCLEOTIDES FOR THE TREATMENT OF DEGENERATIVE SKIN CONDITIONS BY RNA INTERFERENCE

(75) Inventors: Ute Breitenbach, Hamburg (DE); Stefan Gallinat, Wedel (DE); Ludger Kolbe, Dohren (DE); Thomas Blatt, Wedel (DE); Helga Biergiesser, Reinbek (DE); Rainer Wolber, Hamburg (DE); Franz Stab, Echem (DE); Kyra Sanger, Hamburg (DE)

Correspondence Address: GREENBLUM & BERNSTEIN, P.L.C., 1950 Roland Clarke Place, Reston, VA, 20191, US

(73) Assignee: BEIERSDORF, INC.

(21) Appl. No.: 11/850,315

(22) Filed: Sep. 5, 2007

Related U.S. Application Data
(63) Continuation of application No. 11/134,141, filed on May 20, 2005, now abandoned.
Application No. 11/134,141 is a continuation of application No. PCT/EP03/13048, filed on Nov. 20, 2003.

(30) Foreign Application Priority Data
Nov. 20, 2002 (DE) 102 54 214.7

(51) Int. Cl. A61K 31/70; C07H 21/04; C12N 5/00

(52) U.S. Cl. 514/44; 536/22.1; 435/320.1

(57) ABSTRACT

The invention relates to oligoribonucleotides, which are capable of inducing breakdown of the mrna enzymes that break down connective tissue, and to pharmaceutical and cosmetic compositions, which are provided for topical application and which contain the oligoribonucleotides. The compositions are particularly suited for treating degenerative skin disorders.

### CROSS-REFERENCE TO RELATED APPLICATIONS

The present application is a continuation of U.S. application Ser. No. 11/134,141, filed May 20, 2005, which is a continuation of International Application PCT/EP03/13048, filed Nov. 20, 2003.

The invention relates to oligoribonucleotides which induce the decomposition of mRNA of enzymes which decompose connective tissue and in particular are suitable for the treatment and prophylaxis of degenerative skin conditions, such as for example those associated with skin aging.

Chronological skin aging is caused by endogenous, genetically determined factors and manifests itself in age-related structural damage and dysfunctions in the epidermis and dermis of the skin, such as dryness, roughness and development of dry lines/wrinkles, itching and reduced rehydration by sebaceous glands (e.g. after washing). These symptoms are collectively called “senile xerosis”.

Endogenous aging processes can be accelerated and aggravated by exogenous factors such as UV light and chemical noxa. In addition, exogenous influences can cause further structural damage and dysfunctions in the epidermis and dermis of the skin, such as for example visible vascular dilatations (telangiectasis, cuperosis), slackness and formation of wrinkles, local hyper-, hypo- and mispigmentations (e.g. age marks) and increased susceptibility to mechanical stress (e.g. tendency to crack).

Skin aging and wrinkling as a consequence of UV exposure are accompanied by a reduction in skin elasticity and by changes in elastic fibres in the dermis. Histological and ultrastructural studies showed that the biggest changes in skin that had been aged by UV radiation manifest themselves in the connective tissue (Scharffetter-Kochanek K, Wlaschek M, Brenneisen P, Schauen M, Blaudschun R, Wenk J. UV-induced reactive oxygen species in photocarcinogenesis and photoaging. Biol Chem. 1997 November; 378 (11): 1247-57).

Here, the structural damage and dysfunctions caused by exogenous and endogenous factors are called degenerative skin conditions.

Known products for the care of aged skin can contain, in addition to rehydrating constituents, e.g. retinoids (vitamin A acid and/or its derivatives) or vitamin A and/or its derivatives. Tsukahara, K., Y. Takema, et al. describe for example the use of retinoic acid to reduce wrinkling. This is said to effect a regeneration of the elastic fibres (Tsukahara, K., Y. Takema, et al. (2001). “Selective inhibition of skin fibroblast elastase elicits a concentration-dependent prevention of ultraviolet B-induced wrinkle formation.” J Invest Dermatol 117 (3): 671-7).

Active ingredients such as retinol can trigger complex metabolic processes in the cell, vitamin A generally being an initiator for cell regeneration. The substance detaches dead corneocyte cells, replenishes wrinkles from the inside and improves the skin structure.

The effect of these products on structural damage is limited in scope, however. In addition, vitamin A acid-containing products can cause pronounced erythematous skin irritations. Retinoids can therefore be used only in low concentrations. Moreover, there are considerable difficulties during product development in stabilizing the active ingredients sufficiently against oxidative decomposition.

Nor does the use of agents for protection against UV radiation provide extensive protection against degenerative skin changes.

In the literature, the use of tetracyclines and batimastat to inhibit metalloproteinases (MMPs) in cancers is also described. Metalloproteinases play an important part in the decomposition of the connective tissue, in particular the collagen fibres.

Fire et al., Trends Genet. 15 (1999) 358-363 showed that gene expression can be inhibited post-transcriptionally through the presence of double-stranded RNA fragments (dsRNA), which is homologous to the mRNA sequence of the examined gene, and called this process RNA interference (RNAi). In an as yet unexplained manner, dsRNA effects the specific decomposition of the homologous mRNA in the cell and thus prevents protein production.

W001/29058 discloses the identification of genes which participate in RNAi and their use to modulate RNAi activity.

Elbashir et al., Nature 411 (2001) 494-498, describe the specific inhibition of the expression of endogenous and heterologous genes in various mammalian cells by short interfering RNAs, siRNAs. Double-stranded RNA fragments 21 nucleotides long were used.

The reduction of the gene expression in cells by dsRNA is known from W001/68836. dsRNA contains a nucleotide sequence which, under the physiological conditions of the cell, hybridizes with at least a part of the gene to be inhibited. dsRNA is preferably 400 to 800 nucleotides long.

W001/75164 discloses the use of dsRNA 21 to 23 nucleotides long for the specific deactivation of gene functions in mammalian cells by RNAi.

Brummelkamp et al., Science 296 (2002) 550-553, describe a vector system which triggers the synthesis of siRNAs in mammalian cells and is thus said to inhibit the gene expression of a target gene.

EP 1 214 945 A2 discloses the use of dsRNA 15 to 49 base pairs long to inhibit the expression of a preset target gene in mammalian cells. dsRNA can be modified to increase its stability and is said to allow the treatment of cancer, viral diseases and Alzheimer's disease.

W002/053773 relates to an in vitro method for determining skin stress and skin aging in humans and animals, test kits and biochips suitable for carrying out the method and also a test method for demonstrating the effectiveness of cosmetic or pharmaceutical active ingredients against skin stress and skin aging.

Oligoribonucleotides which are suitable for the treatment of degenerative skin conditions have not been described to date.

The object of the present invention is the provision of compositions which make possible an effective treatment of degenerative skin states and in particular skin states due to aging, without displaying the disadvantages of the state of the art.

This object is achieved by oligoribonucleotides which are capable of inhibiting the expression of genes of enzymes which decompose connective tissue.

By enzymes which decompose connective tissue are meant primarily peptidases, in particular endopeptidases such as collagen- and elastin-decomposing endopeptidases, and glycosaminoglycan-decomposing enzymes, in particular hyaluronic acid-decomposing endo-N-acetylglucosaminidases, in particular hyaluronidases. Hyaluronic acid is also called hyaluronane.

In addition to the named oligoribonucleotides, physiologically compatible salts of such oligoribonucleotides are also suitable according to the invention. For simplicity's sake, the term oligoribonucleotide will be used hereafter for both the actual oligoribonucleotides and for their salts, unless otherwise stated. The term oligoribonucleotide also includes modified oligoribonucleotides.

Preferred endopeptidases include primarily collagen-decomposing and elastin-decomposing endopeptidases, in particular matrix metalloproteinases (MMPs) and elastases. Preferred MMPs include the following enzymes which can be divided into collagenases and non-collagenases:

| | | |
|---|---|---|
| MMP-1 | P03956 | (EC 3.4.24.7) |
| MMP-2 | P08253 | (EC 3.4.24.24) |
| MMP-3 | P08254 | (EC 3.4.24.17) |
| MMP-7 | P09237 | (EC 3.4.24.23) |
| MMP-8 | P22894 | (EC 3.4.24.34) |
| MMP-9 | P14780 | (EC 3.4.24.35) |
| MMP-10 | P09238 | (EC 3.4.24.22) |
| MMP-11 | P24347 | (EC 3.4.24) |
| MMP-12 | P39900 | (EC 3.4.24.65) |
| MMP-13 | P45452 | (EC 3.4.24) |
| MMP-14 | P50281 | (EC 3.4.24) |
| MMP-15 | P51511 | (EC 3.4.24) |
| MMP-16 | P51512 | (EC 3.4.24) |
| MMP-17 | Q9ULZ9 | (EC 3.4.24) |
| MMP-19 | Q99542 | (EC 3.4.24) |
| MMP-20 | 060882 | (EC 3.4.24) |
| MMP-24 | Q9Y5R2 | (EC 3.4.24) |
| MMP-25 | Q9NPA2 | (EC 3.4.24) |
| MMP-26 | Q9NRE1 | (EC 3.4.24) |
| MMP-28 | Q9H239 | (EC 3.4.24) |

The enzymes MMP 1, 8 and 13 are collagenases, the other named enzymes non-collagenases. The numbers given are the accession numbers of the Swiss PROT EMBL-EBI database (European Bioinformatics Institute Heidelberg).

Preferred elastases include the enzymes which are isolated from the pancreas, from macrophages and from leukocytes, in particular the enzyme ELA2 (M34379 EC 3.4.21.37).

Preferred endo-N-acetylglucosaminidases include:

| | |
|---|---|
| SPAM1 | (s67798) |
| HYAL3 | (AF036035) |
| HYAL4 | (AF009010) |
| HYAL5 | (AF036144) |

and in particular HYAL2 (U09577). The accession numbers given here are those of the NCBI database (National Center for Biotechnology Information) of the National Institute of Health.

Collagen-decomposing endopeptidases (collagenases) are enzymes which degrade the structure proteins of the connective tissue and are responsible for the decomposition of elastin and collagen fibres, but also of proteoglycans. The controlled activity of these enzymes plays a decisive role in tissue restructuring during development, tissue repair and angiogenesis processes.

Oligoribonucleotides are quite particularly preferred which can inhibit the expression of zinc-dependent endopeptidases (matrix metalloproteinases, MMPs), in particular the matrix metalloproteinases 1, 8 and 13, quite particularly preferably the matrix metalloproteinase 1. These enzymes are described e.g. in Fisher G J, Choi H C, Bata-Csorgo Z, Shao Y, Datta S, Wang Z Q, Kang S, Voorhees J J., Ultraviolet irradiation increases matrix metalloproteinase-8 protein in human skin in vivo, J Invest Dermatol. 2001 August; 117(2):219-26.

Oligoribonucleotides which can inhibit the expression of the mRNA of the matrix metalloproteinase 9 are equally preferred. It is assumed that this, together with the metalloproteinases 1, 8 and 13, is involved in the process caused by UV radiation, of the so-called “photoaging” of the skin.

According to the invention, compositions are also particularly preferred which contain oligoribonucleotides which are capable of inhibiting the expression of serin proteinases, such as pancreatic and neutrophilic elastases and macrophage elastases, which belong to the group of elastases.

From the mechanistic point of view, elastases (pancreatic and neutrophilic elastases, macrophage-elastase) play an important role in the degeneration of elastic fibres. These serin proteinases participate among other things in phagocytotic processes, in defense against microorganisms, the degradation of elastin, collagens, proteoglycans, fibrinogen and fibrin and tissue damaged during digestion (Bolognesi, M., K. Djinovic-Carugo, et al. (1994). “Molecular bases for human leucocyte elastase inhibition.” Monaldi Arch Chest Dis 49 (2): 144-9).

In particular, neutrophilic elastase is accorded great significance in the development of solar elastosis (Starcher, B. and M. Conrad (1995). “A role for neutrophil elastase in solar elastosis.” Ciba Found Symp 192: 338-46; discussion 346-7). Biochemical studies have shown that human dermal fibroblasts from skin with dermal elastosis have high levels of elastase and cathepsin G (Fimiani, M., C. Mazzatenta, et al. (1995). “Mid-dermal elastolysis: an ultrastructural and biochemical study.” Arch Dermatol Res 287 (2): 152-7).

Compositions also particularly preferred according to the invention are those which contain oligonucleotides which are capable of hybridizing with the genes or mRNAs of hyaluronidases, preferably the already named enzymes SPAM1 (s67798), HYAL3 (AF036035), HYAL4 (AF009010), HYAL5 (AF036144) and particularly preferably HYAL2 (U09577).

Oligoribonucleotides are also preferred according to the invention which inhibit the expression of proteinases, in the particular the enzymes named below. The accession numbers given are from the UniGene database which can also be accessed via the NCBI database: Hs. 274404 (PLAT plasminogen activator, tissue); Hs. 179657 (PLAUR plasminogen activator, urokinase receptor, Homo sapiens); Hs. 77274 (PLAU plasminogen activator, urokinase, Homo sapiens); Hs. 169172 (CAPN6 calpain 6, Homo sapiens); Hs. 76288 (CAPN2 calpain 2, (m/II) large subunit, Homo sapiens); Hs. 7145 (CAPN7 calpain 7, Homo sapiens); Hs. 2575 (CAPN1 calpain 1, (mu/I) large subunit, Homo sapiens); Hs. 6133 (CAPN5 calpain 5, Homo sapiens); Hs. 55408 (CAPNS2 calpain small subunit 2, Homo sapiens); Hs. 211711 (ESTs, Weakly similar to CAN1 HUMAN Calpain 1, large [catalytic] subunit (Calcium-activated neutral proteinase) (CANP) (Mu-type) (muCANP) (Micromolar-calpain) [H. sapiens], Homo sapiens); Hs. 112218 (CAPN10 calpain 10, Homo sapiens); Hs. 74451 (CAPNS1 calpain, small subunit 1); Hs. 225953 (CAPN11 calpain 11, Homo sapiens); Hs. 113292 (CAPN9 calpain 9 (nCL-4), Homo sapiens); Hs. 387705 (CAPN13 calpain 13, Homo sapiens); Hs. 297939 (CTSB cathepsin B, Homo sapiens); Hs. 343475 (CTSD cathepsin D (lysosomal aspartyl protease); Homo sapiens); Hs. 83942 (CTSK cathepsin K (pycnodysostosis), Homo sapiens); Hs. 78056 (CTSL cathepsin L, Homo sapiens); Hs. 181301 (CTSS cathepsin S, Homo sapiens).

The oligoribonucleotides according to the invention are RNA molecules (RNAs) which fully or partially suppress the expression of these enzymes (gene switch-off, gene silencing), which is presumably attributable to the decomposition of the mRNA of one of the above-named enzymes. This process is called RNA interference (RNAi). The invention thus relates to oligoribonucleotides which can induce the decomposition of the mRNA of enzymes which decompose connective tissue. The mRNA the decomposition of which is to be effected is also called target mRNA below. Accordingly, by target gene is meant the gene and in particular the coding region of the gene the expression of which is fully or partially suppressed. Unless otherwise indicated, the term target sequence refers to both the target gene and the target mRNA. The decomposition of the mRNA of enzymes which decompose connective tissue by RNAi is sequence-specific, i.e. as a rule an oligoribonucleotide inhibits only the expression of the corresponding target gene.

The coding regions (cDNA) of the respective genes are preferred as target sequence for the oligoribonucleotides according to the invention, including the 5′ and 3′ UTR regions. The areas of the coding regions which lie 50 to 100 nucleotides downstream of the start codon are particularly preferred.

The oligoribonucleotides according to the invention are preferably double-stranded RNA molecules (dsRNAs) which are homologous to the sequence of the target gene or a section of it, i.e. are identical to the target gene as regards sense and antisense strands.

According to the invention, homology is also given when the dsRNA is not completely identical to the target sequence. Relative to a length of 20 base pairs, the oligoribonucleotides according to the invention preferably display a maximum of 0 to 2, particularly preferably 0 to 1 and quite particularly preferably no deviations from the target sequence, i.e. at most 0 to 2 and in particular at most 0 to 1 base pairs are replaced by other base pairs.

The oligoribonucleotides according to the invention are preferably 15 to 49 nucleotides, preferably 17 to 30, particularly preferably 19 to 25 and quite particularly preferably 20 to 23 nucleotides long.

However, a subject of the invention are also longer nucleotide fragments such as e.g. dsRNAs which correspond in length to the respective mRNAs or cDNAs. These can be transformed, e.g. by soluble Drosophila embryo extract, into fragments 21 to 23 nucleotides long (cf. W001/75164). Long-chained dsRNA is also decomposed intracellularly into short pieces. However, the direct use of long-chained dsRNA is in general not preferred as it can effect an unspecific inhibition of translation in mammalian cells.

The RNA duplexes according to the invention can have blunt or overhanging (sticky) ends. Double-stranded oligoribonucleotides have proved particularly effective which have an overhang of 1 to 6, preferably 1 or 2 nucleotides, at the 3′ end of each strand. The overhanging nucleotides are preferably 2-desoxynucleotides, particularly preferably 2-desoxythymidine residues. The costs of the RNA synthesis can be reduced, and the resistance of the RNA to nuclease decomposition increased by using 2-desoxynucleotides. The overhanging nucleotides need not necessarily be nucleotides homologous to the target sequence and are therefore not taken into account in the deviations defined above from the target sequence. However, oligoribonucleotides with short overhangs, in particular of 2 nucleotides, in which the overhanging nucleotides of the antisense strand of dsRNA are complementary to the target sequence, are preferred.

Oligoribonucleotides have proved particularly effective which are homologous to such a section of the target gene and in particular the corresponding double-stranded cDNA the sense strand of which is delimited at the 5′ side by two adenosine radicals (A) and at the 3′ side by two thymidine radicals (T) or one thymidine and one cytidine radical (C). The section delimited by AA and TT or AA and TC is preferably 19 to 21, in particular 19 nucleotides long and accordingly has the general form AA(N₁₉₋₂₁)TT or AA(N₁₉₋₂₁)TC, N standing for a nucleotide. Further preferred are oligoribonucleotides which are complementary to a section of the target gene or the corresponding double-stranded cDNA which has the general form AA(N₁₉) to AA(N₂₁). Oligoribonucleotides which are homologous to the N₁₉₋₂₁ fragment of the named regions are particularly preferred. The particularly preferred oligoribonucleotides are thus 19 to 21 base pairs long, the single strands forming these oligoribonucleotides preferably each having two additional 2′-desoxynucleotides, in particular two 2-desoxythymidine radicals at the 3′ side with the result that the dsRNA comprises 19 to 21 base pairs and two overhanging 2-desoxynucleotides per strand.

Should the target gene not contain a region of the form AA(N₁₉₋₂₁), regions of the form NA(N₁₉₋₂₁) or any fragment of the form N₁₉₋₂₁ are sought. Although N₁₉₋₂₁ fragments which are delimited e.g. by AA and TT are preferable, in principle according to the invention all dsRNA fragments which are homologous to the target sequence are suitable.

### BRIEF DESCRIPTION OF THE DRAWINGS

FIG. 1 shows the single-stranded cDNA of the matrix metalloproteinase 1 (SEQ ID No. 1) in which all fragments of the form AA-N₁₉-TT and AA-N₁₉-TC are highlighted.

FIG. 2 shows the fragments (targeted regions) which are highlighted in FIG. 1 together with the corresponding homologous and complementary RNA single strands.

FIG. 3 shows the single-stranded cDNA of elastase 2 (SEQ ID No. 59).

FIG. 4 shows the single-stranded cDNA of hyaluronidase 2 (SEQ ID No. 61) with preferred sequence regions marked.

FIG. 5 graphically represents the expression of MMP-1 by HeLaS3 cells obtained according to Example 1 below.

FIG. 6 graphically represents the expression of MMP-1 by HeLaS3 cells relative to control Lamin A/C obtained according to Example 9 below.

FIG. 7 graphically represents the expression of MMP-1 by female primary fibroblasts obtained according to Example 10 below.

FIG. 8 graphically represents the expression of MMP-1 by male primary fibroblasts obtained according to Example 11 below.

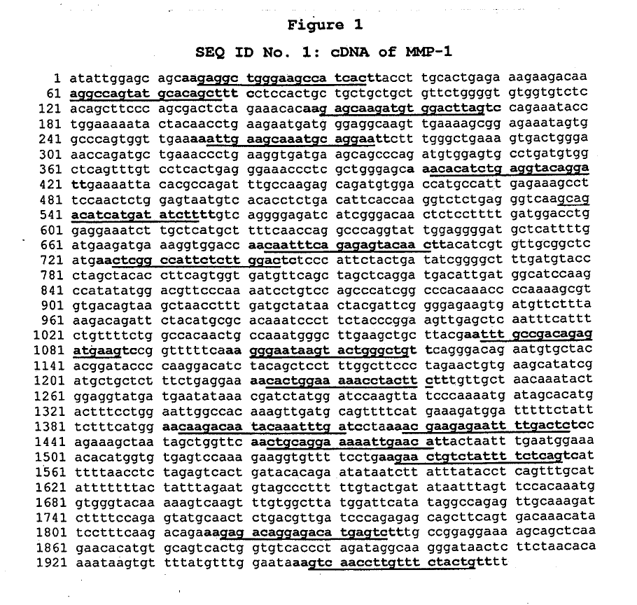

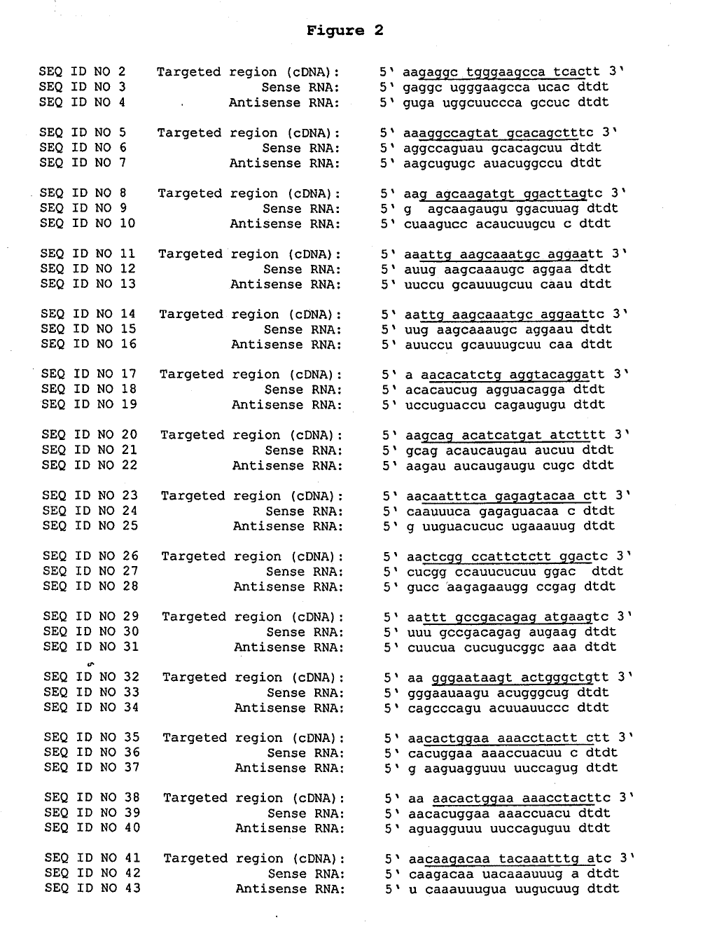

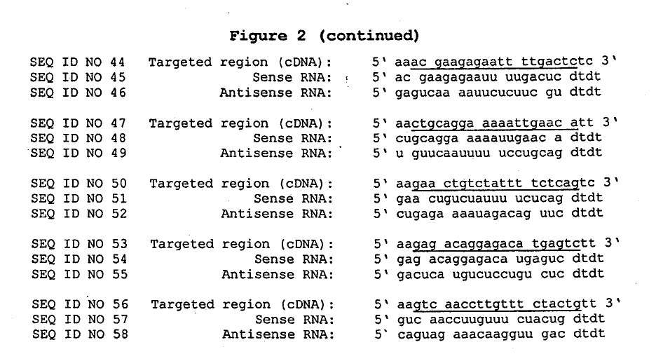

FIG. 1 shows the single-stranded cDNA of the matrix metalloproteinase 1 (SEQ ID No. 1) in which all fragments of the form AA-N₁₉-TT and AA-N₁₉-TC are highlighted. In FIG. 2 these fragments (targeted region) are shown together with the corresponding homologous (sense RNA) and complementary (antisense RNA) RNA single strands. Single-stranded RNAs which are modified at the 3′ side by two desoxythymidine radicals (dt) are shown. The hybridization of two complementary single-stranded RNAs results in dsRNA with overhanging 3′ ends each of which is formed by two 2′-desoxythymidine radicals.

The gene of the matrix metalloproteinase 1 is among the preferred target genes for the oligoribonucleotides according to the invention. Oligoribonucleotides which are homologous to the double-stranded sequence derived from SEQ ID No. 1, sections thereof and in particular the double-stranded sequences which are derived from the sections highlighted in FIG. 1, are accordingly particularly preferred according to the invention. By the double-stranded sequence derived from SEQ ID No. 1 is meant the sequence which is formed from SEQ ID No. 1 and the strand complementary to it. The other details are to be understood accordingly. Oligoribonucleotides which are homologous to the region from position 601 to 1441 of SEQ ID No. 1 are particularly preferred, those which are homologous to the region from position 1099 to 1121 quite particularly preferred.

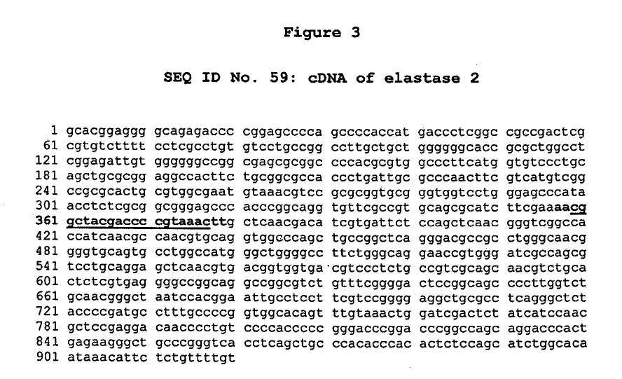

The single-stranded cDNA of elastase 2 (SEQ ID No. 59) can be seen in FIG. 3. Here also, a preferred sequence region, i.e. a sequence region 19 nucleotides long which is flanked by AA and TT, is highlighted. Oligoribonucleotides which are homologous to the double-stranded sequence derived from SEQ ID No. 59, sections thereof and in particular the double-stranded sequence which is derived from the region highlighted in FIG. 2 are likewise preferred according to the invention.

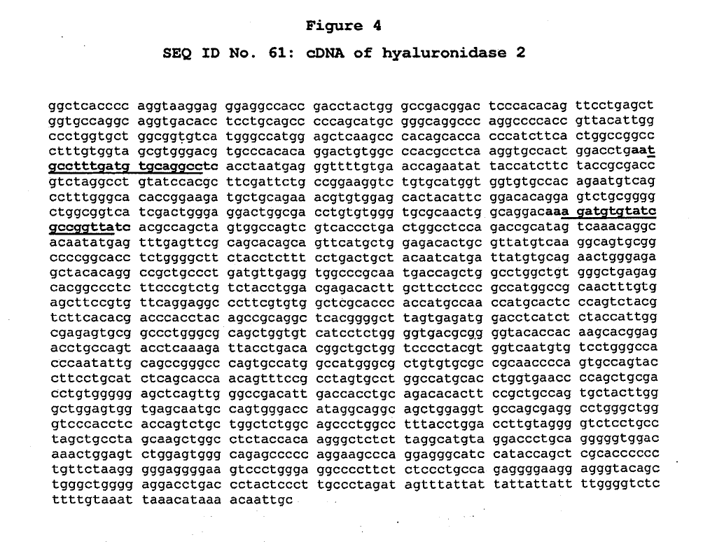

FIG. 4 shows the single-stranded cDNA of hyaluronidase 2 (SEQ ID No. 61), preferred sequence regions again being marked. Oligoribonucleotides which are homologous to the double-stranded sequence derived from SEQ ID No. 61, sections thereof and in particular the double-stranded sequence which is derived from the region highlighted in FIG. 3 are likewise preferred according to the invention.

The oligoribonucleotides according to the invention can advantageously also be integrated into expression vectors, in particular those which effect an expression of the oligoribonucleotides in mammalian cells. In this way, even in the case of an intracellular decomposition of the oligoribonucleotides, a stable inhibition of the expression of the target gene can be achieved, as oligoribonucleotides are continuously re-delivered through the vector-supported synthesis. One or more copies of a dsRNA, but also one or more copies each of two or more different dsRNAs can be integrated into a vector. Suitable vector systems are described e.g. by Brummelkamp et al., loc cit. Mammalian expression vectors are preferred, in particular those which contain a polymerase III-Hl RNA promoter and 5 to 9 so-called loops which are formed from a dsRNA according to the invention and a sequence of equal length which is reverse complementary to the dsRNA according to the invention and serves as spacer, and a termination signal of 5 successive thymidine radicals. The vectors thus contain 5 to 9 copies of the respective dsRNA molecule. These can be dsRNAs which are specific to 1 target gene or dsRNAs which are specific to several different target genes.

The oligoribonucleotides according to the invention can be present in the form of the unmodified oligoribonucleotides. However, they are preferably oligoribonucleotides which can be chemically modified on the level of the sugar radicals, the nucleobases, the phosphate groups and/or the backbone located in between, in order to increase for example the stability of the oligoribonucleotides in the cosmetic or dermatological preparations and/or in the skin, e.g. vis-à-vis a nucleolytic decomposition, in order to improve the penetration of the oligoribonucleotides into the skin and the cell, in order to favourably influence the effectiveness of the oligoribonucleotides and/or to improve the affinity to the sequence sections to be hybridized.

Oligoribonucleotides are preferred in which one or more phosphate groups are replaced by phosphothioate, methylphosphonate and/or phosphoramidate groups, such as e.g. N3′->P5′-phosphoramidate groups. Oligoribonucleotides in which phosphate groups are replaced by phosphothioate groups are particularly preferred. One or more of the phosphate groups of the oligoribonucleotide can be modified. In the case of a partial modification, terminal groups are preferably modified, but oligoribonucleotides in which all the phosphate groups are modified are particularly preferred. This applies by analogy also to the modifications described below.

Preferred sugar modifications include the replacement of one or more ribose radicals of the oligoribonucleotide by morpholine rings (morpholine oligoribonucleotides) or by amino acids (peptide oligoribonucleotides). All ribose radicals of the oligoribonucleotide are preferably replaced by amino acid radicals and in particular morpholine radicals. Morpholine oligoribonucleotides are particularly preferred in which the morpholine radicals are connected to one another via sulfonyl or preferably phosphoryl groups, as can be seen in Formula 1 or 2:

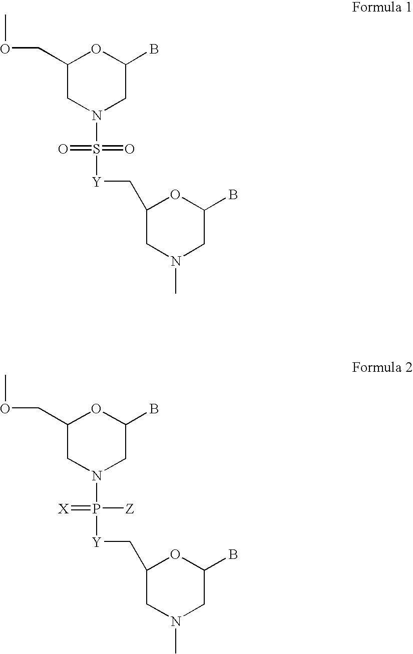

B stands for a modified or non-modified purine or pyrimidine base, preferably for adenine, cytosine, guanine or uracil,
X stands for O or S, preferably O,
Y stands for O or N—CH₃, preferably O,
Z stands for alkyl, O-alkyl, S-alkyl, NH₂, NH(alkyl), NH(O-alkyl), N(alkyl)₂, N(alkyl)(O-alkyl), preferably N(alkyl)₂, alkyl standing for linear or branched alkyl groups with 1 to 6, preferably 1 to 3, and particularly preferably 1 or 2 carbon atoms.

Formulae 1 and 2 each represent only a section of an oligoribonucleotide chain.

Morpholine oligoribonucleotides are quite particularly preferred in which the morpholine radicals are connected to one another via phosphoryl groups, as shown in Formula 2, in which X stands for O, Y for O and Z for N(CH₃)₂.

Furthermore, the ribose radicals can be modified by amino, such as NH₂, fluorine, alkyl or O-alkyl radicals, such as OCH₃, 2′-modified oligoribonucleotides being particularly preferred. Examples of modifications are 2′-fluoro, 2′-alkyl, 2′-O-alkyl, 2′-O-methoxyethyl modifications, 5′-palmitate derivatives and 2′-O-methylribonucleotides.

The modification of the nucleotides of dsRNA acts in the cells against an activation of the protein kinase PKR which depends on double-stranded RNA. As a result, an unspecific inhibition of translation is prevented. In particular the substitution of at least one 2′-hydroxyl group of the nucleotides of the dsRNA by a 2′-amino or one 2′-methyl group is suitable for this purpose. Furthermore at least one nucleotide in at least one strand of the dsRNA can be replaced by a so-called “locked nucleotide” which contains a chemically modified sugar ring. A preferred modification of the sugar ring is a 2′-0, 4′-C methylene bridge. dsRNA which contains several “locked nucleotides” is preferred.

Unless otherwise stated, alkyl preferably stands here for linear, branched or cyclic alkyl groups with 1 to 30, preferably 1 to 20, particularly preferably 1 to 10 and quite particularly preferably 1 to 6 carbon atoms. Branched and cyclic radicals naturally have at least 3 carbons, cyclic radicals with at least 5 and in particular at least 6 carbon atoms being preferred.

Oligoribonucleotides which contain α-nucleosides can equally be used.

Suitable base modifications are described e.g. in U.S. Pat. No. 6,187,578 and WO 99/53101, which are incorporate herein by reference. A modification of one or more pyrimidines in position 5 with I, Br, Cl, NH₃ and N₃ has proved advantageous.

The synthesis of modified and non-modified oligoribonucleotides as well as further suitable possible modifications are described in the literature. The production of modified and non-modified oligoribonucleotides is now also offered by numerous companies as a service, for example by the companies Dharmacon, 1376 Miners Drive#101, Lafayette, Colo. 80026, USA, Xeragon Inc., Genset Oligos and Ambion. The preparation of oligoribonucleotides is also described in U.S. Pat. No. 5,986,084.

To increase stability and/or penetration, the oligoribonucleotides can also be used in encapsulated form, for example encapsulated in liposomes. In addition, they can also be stabilized by the addition of cyclodextrins.

Cyclodextrins are also called cycloamyloses and cycloglucans. Cyclodextrins are cyclic oligosaccharides consisting of α-1,4 linked glucose units. As a rule, six to eight glucose units (α-, β-, or γ-cyclodextrins) are linked together. Cyclodextrins are obtained by the action of Bacillus macerans on starch. They are hydrophobic on the inside and hydrophilic on the outside. Both the cyclodextrins themselves, in particular α-cyclodextrin, β-cyclodextrin and γ-cyclodextrin, and derivatives thereof are suitable according to the invention.

According to the invention, the cyclodextrin or cyclodextrins can be used in cosmetic and dermatological compositions, preferably in a concentration of 0.0005 to 20.0 wt.-%, in particular 0.01 to 10 wt.-% and particularly preferably in a concentration of 0.1 to 5.0 wt.-%.

It is advantageous according to the invention to use native, polar and/or non-polar-substituted cyclodextrins. These preferably but not exclusively include methyl, in particular random methyl-β-cyclodextrin, ethyl and also hydroxypropyl cyclodextrins, for example hydroxypropyl-β-cyclodextrin and hydroxypropyl-γ-cyclodextrin. The cyclodextrin species particularly preferred according to the invention are γ-cyclodextrin and hydroxypropyl-β-cyclodextrin.

Liposomes can be prepared in per se known manner using natural phospholipids, such as e.g. phosphatidylcholine from eggs, soybeans etc., or synthetic phospholipids (cf. G. Betageri (editor), “Liposome Drug Delivery Systems”, Lancaster Techonomic Publishing Company 1993; Gregoriadis (editor), “Liposome Technology”, CRC Press). Preferred processes and materials for the preparation of liposomes are described in WO 99/24018.

Double-stranded oligoribonucleotides can also be modified in order to counter a dissociation into the single strands, for example by one or more covalent, coordinative or ionic bonds. Oligoribonucleotides without such modifications are preferred, however.

The nucleotides in the RNA molecules can also comprise “non-standard” nucleotides such as e.g. nucleotides or desoxyribonucleotides which do not occur naturally.

Oligoribonucleotides are preferred according to the invention which inhibit the expression of the respective target gene compared with untreated cells by at least 40%, preferably by at least 60%, particularly preferably by at least 80% and quite particularly preferably by at least 85%. If necessary, the expression of the target gene is firstly induced in suitable manner in the cells in order to measure the inhibition. Tumoral cells of the HeLaS3 line are preferably used to determine the effectiveness of the oligoribonucleotides according to the invention. The oligoribonucleotides are introduced into the cells and then, optionally after induction of the expression of the target gene, the expression rate of the target gene in these cells is measured and compared with that which is found in cells which have not been transfected with the oligoribonucleotide concerned. The exact conditions for measuring the inhibition are found in Example 1.

The oligoribonucleotides according to the invention and their salts are particularly suitable as an effective constituent of pharmaceutical and cosmetic compositions, in particular those for topical application.

It was surprisingly found that the oligoribonucleotides, following application of the compositions to the skin, inhibit the expression of the genes which are responsible for the decomposition of the connective tissue and thus prevent the degeneration of collagen, elastin and/or hyaluronic acid without side-effects and in this way make possible an effective treatment and prophylaxis of degenerative skin conditions without displaying the disadvantages of the state of the art. It is assumed that this effect is attributable to the fact that the oligoribonucleotides according to the invention are absorbed by the cells of the skin and intracellularly induce the decomposition of the mRNAs of the named genes by RNAi, details of the mechanism of this reaction cascade are not yet known. The oligoribonucleotides are therefore particularly suitable for initiating the decomposition of mRNA of enzymes which decompose connective tissue and for inhibiting the expression of enzymes which decompose connective tissue in the skin and in particular in skin cells.

The compositions according to the invention can also contain one or more oligoribonucleotides which inhibit the expression of the protein kinase PKR and thus counter an unspecific inhibition of translation.

The pharmaceutical or cosmetic compositions according to the invention preferably contain 0.00001 to 10 wt.-%, particularly preferably 0.0003 to 3 wt.-% and quite particularly preferably 0.01 to 1.0 wt.-% of the oligoribonucleotide or oligoribonucleotides according to the invention, relative to the overall mass of the composition. When using oligoribonucleotides which are integrated into vectors, the quantity given above relates to the mass of the oligoribonucleotides integrated into the vector, the mass of the vector itself not being taken into account.

Compositions are preferred according to the invention which contain exclusively oligoribonucleotides which inhibit the expression of one or more of the genes named above, i.e. the genes of enzymes which decompose connective tissue and optionally of proteinase PKR, and in particular the named preferred genes. The compositions according to the invention can contain one or preferably several oligoribonucleotides. These can be oligoribonucleotides which inhibit the expression of several different collagen-decomposing enzymes, elastases and/or hyaluronidases, but mixtures of oligoribonucleotides can also be used which target different sequence regions of one and the same gene or the same mRNA of a collagen-decomposing enzyme, an elastase and/or a hyaluronidase. Compositions which contain 1 to 5 and in particular 1 to 3 different oligoribonucleotides are preferred. Mixtures of oligoribonucleotides which unspecifically inhibit or induce the activity of a plurality of different skin proteins in addition to the named enzymes which decompose connective tissue and optionally the proteinase PKR are undesired, as almost no monitoring of side-effects is possible. By skin proteins are meant proteins which are expressed in the skin. Compositions are quite particularly preferred which contain one or several oligoribonucleotides which inhibit the expression of one or more hyaluronidases.

Compositions are also particularly preferred which each contain at least one oligoribonucleotide which is directed against a collagen-decomposing enzyme, an elastase and a hyaluronidase.

The oligoribonucleotides and compositions are suitable for the treatment and prophylaxis of aging- and environmentally-triggered degenerative and deficitary conditions of the skin and of skin adnexa, such as hair and glands, in particular the symptoms described above. They are suitable for the cosmetic and therapeutic treatment of degenerative skin conditions which are caused by endogenous and exogenous factors, such as ozone and smoking and in particular UV radiation. The compositions according to the invention can prevent skin damage and repair existing damage permanently and without the risk of side-effects. The method described in W002/053773 for example can be used to determine the effectiveness of the oligoribonucleotides according to the invention.

The oligoribonucleotides according to the invention are particularly suitable for the prevention and treatment of age-related skin changes and skin changes which are caused by UV radiation in the connective tissue, such as e.g. skin changes which accompany biochemical, quantitative or qualitative changes in different dermal, extracellular proteins, in particular elastin, interstitial collagen and glycosaminoglycans. Wrinkling, slackness of the skin, loss of elasticity and mispigmentations (e.g. age marks) may primarily be named here.

The oligoribonucleotides and compositions are suitable for the prophylaxis and treatment of dryness, roughness of the skin, the formation of dry lines, reduced rehydration by sebaceous glands and an increased susceptibility to mechanical stress (tendency to crack), for the treatment of photodermatoses, the symptoms of senile xerosis, photoaging and other degenerative conditions which are associated with a decomposition of the connective tissue (collagen and elastin fibres and also glucosaminoglycans/hyaluronane) of the skin. “Photoaging” denotes the wrinkling, dryness and decreasing elasticity of the skin brought about by light and in particular UV radiation.

Due to their prophylactic action, the oligoribonucleotides and compositions according to the invention are also outstandingly suitable for care of the skin.

The compositions according to the invention are also suitable for the treatment of skin damage caused by UV rays, e.g. the ultraviolet portion of solar radiation. UVB rays (290 to 320 nm) cause for example erythemas, sunburn or even burns of greater or lesser severity. UVA rays (320 to 400 nm) can cause irritations in light-sensitive skin and result in damage to the elastic and collagen fibres of the connective tissue, which causes the skin to age prematurely. In addition they are the cause of numerous phototoxic and photoallergic reactions. The oligoribonucleotides according to the invention are also suitable for the treatment of e.g. structural damage caused by UV rays and dysfunctions in the epidermis and dermis of the skin, such as for example visible vascular dilatations, such as telangiectasis and cuperosis, slackness of the skin and formation of wrinkles, local hyper-, hypo- and mispigmentations, such as e.g. age marks, and increased susceptibility to mechanical stress, e.g. tendency of the skin to crack.

Further fields of application of the compositions according to the invention are the treatment and prevention of age- and/or UV-induced collagen degeneration and also the decomposition of elastin and glycosaminoglycans; of degenerative skin conditions such as loss of elasticity and also atrophy of the epidermal and dermal cell layers, of constituents of the connective tissue, of rete pegs and capillary vessels) and/or the skin adnexa; of environmentally-triggered negative changes in the skin and the skin adnexa, e.g. caused by ultraviolet radiation, smoking, smog, reactive oxygen species, free radicals and similar; of deficitary, sensitive or hypoactive skin conditions or deficitary, sensitive or hypoactive skin adnexa conditions; the reduction in skin thickness; of skin slackness and/or skin tiredness; of changes in the transepidermal water loss and normal moisture content of the skin; of a change in the energy metabolism of healthy skin; of deviations from the normal cell-cell communication in the skin which can manifest themselves e.g. in wrinkling; of changes in the normal fibroblast and keratinocyte proliferation; of changes in the normal fibroblast and keratinocyte differentiation; of polymorphic actinodermatosis, vitiligo; of wound healing disorders; disturbances to the normal collagen, hyaluronic acid, elastin and glycosaminoglycan homeostasis; of increased activation of proteolytic enzymes in the skin, such as e.g. metalloproteinases.

According to the invention, compositions for topical applications are preferred. The compositions can be in all galenic forms which are usually used for a topical application, e.g. as solution, cream, ointment, lotion, shampoo, i.e. of the water-in-oil (W/O) emulsion type or of the oil-in-water (O/W) type, multiple emulsion, for example of the water-in-oil-in-water (W/O/W) type, or oil-in-water-in-oil (O/W/O) type, hydrodispersion or lipodispersion, Pickering emulsion, gel, stick or aerosol.

The cosmetic or medical treatment of the named indications is carried out as a rule by single or repeated application of the compositions according to the invention to the skin, preferably to the affected parts of the skin.

The compositions according to the invention are suitable in particular for cosmetic and therapeutic, i.e. in particular dermatological, application.

By cosmetic care of the skin is meant primarily that the natural function of the skin as a barrier against environmental influences (e.g. dirt, chemicals, microorganisms) and against the loss of the body's own substances (e.g. water, natural fats, electrolytes) is reinforced or restored. If this function is disrupted, increased resorption of toxic or allergenic substances or attack by microorganisms and consequently toxic or allergic skin reactions may result. The aim of skin care is further to compensate for the fat and water lost by the skin due to daily washing. This is important precisely when the natural regeneration capacity is insufficient. In addition, skin care products are to protect against environmental influences, in particular against sun and wind.

For cosmetic application, the compositions according to the invention therefore preferably contain components which are suitable for the named purposes. Such substances are known per se to a person skilled in the art. For example, one or more antisense oligoribonucleotides can be incorporated into customary cosmetic and dermatological preparations, and can be present in various forms.

According to a particularly preferred version, the compositions according to the invention for cosmetic application are present as emulsion, e.g. in the form of a cream, a lotion, a cosmetic milk. These contain, in addition to the named oligoribonucleotides, further components such as e.g. fats, oils, waxes and/or other fatty bodies, plus water and one or more emulsifiers such as are usually used for such a formulation type.

As a rule, emulsions contain a lipid or oil phase, an aqueous phase and preferably also one or more emulsifiers. Compositions are particularly preferred which also contain one or more hydrocolloids.

The compositions according to the invention preferably contain 0.001 to 35 wt.-%, particularly preferably 2 to 15 wt.-% emulsifier, 0.001 to 45 wt.-%, particularly preferably 10 to 25 wt.-% lipid and 10 to 95 wt.-%, particularly preferably 60 to 90 wt.-% water.

The lipid phase of the cosmetic or dermatological emulsions according to the invention can advantageously be chosen from the following substance group: (1) mineral oils, mineral waxes; (2) oils such as triglycerides of capric or caprylic acid, also natural oils such e.g. castor oil; (3) fats, waxes and other natural and synthetic fatty bodies, preferably esters of fatty acids with alcohols of low C number, e.g. with isopropanol, propylene glycol or glycerol, or esters of fatty alcohols with alkanoic acids of low C number or with fatty acids; (4) alkyl benzoates; (5) silicone oils such as dimethylpolysiloxanes, diethylpolysiloxanes, diphenylpolysiloxanes and also mixed forms thereof.

Unless otherwise stated, by low C number is meant here preferably 1 to 5, particularly preferably 1 to 3 and quite particularly preferably 3 carbon atoms.

The oil phase of the emulsions of the present invention is advantageously chosen from the group of esters from saturated and/or unsaturated, branched and/or unbranched alkane carboxylic acids of a chain length of 3 to 30 C atoms and saturated and/or unsaturated, branched and/or unbranched alcohols of a chain length of 3 to 30 C atoms, from the group of esters from aromatic carboxylic acids and saturated and/or unsaturated, branched and/or unbranched alcohols of a chain length of 3 to 30 C atoms. Such ester oils can advantageously be chosen from the group isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and also synthetic, semi-synthetic and natural mixtures of such esters, e.g. jojoba oil.

Furthermore the oil phase can advantageously be chosen from the group of branched and unbranched hydrocarbons and waxes, silicone oils, dialkyl ethers, the group of saturated or unsaturated, branched or unbranched alcohols, and also the fatty acid triglycerides, namely the triglycerol esters of saturated and/or unsaturated, branched and/or unbranched alkane carboxylic acids of a chain length of 8 to 24, in particular 12-18 C atoms. The fatty acid triglycerides can for example advantageously be chosen from the group of synthetic, semi-synthetic and natural oils, e.g. olive oil, sunflower oil, soya oil, peanut oil, rape-seed oil, almond oil, palm oil, coconut oil, palm-kernel oil and more of this kind.

Any desired mixtures of such oil and wax components are also advantageously to be used within the meaning of the present invention. It may also be advantageous where appropriate to use waxes, for example cetyl palmitate, as sole lipid component of the oil phase.

The oil phase is advantageously chosen from the group 2-ethylhexyl isostearate, octyl dodecanol, isotridecyl isononanoate, isoeicosane, 2-ethylhexyl cocoate, C₁₂₋₁₅ alkylbenzoate, caprylic-capric acid triglyceride, dicaprylyl ether.

Mixtures of C₁₂₋₁₅ alkylbenzoate and 2-ethylhexyl isostearate, mixtures of C₁₂₋₁₅ alkylbenzoate and isotridecyl isononanoate and also mixtures of C₁₂₋₁₅ alkylbenzoate, 2-ethylhexyl isostearate and isotridecyl isononanoate are particularly advantageous.

Of the hydrocarbons, paraffin oil, squalane and squalene are advantageously to be used within the meaning of the present invention.

The oil phase can advantageously also contain cyclic or linear silicone oils or consist entirely of such oils, it being preferred however to use an additional content of other oil phase components in addition to the silicone oil or silicone oils. Such silicones or silicone oils can be present as monomers which are characterized as a rule by structural elements, as follows:

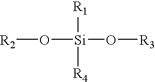

Linear silicones to be used advantageously according to the invention with several siloxyl units are in general characterized by structural elements as follows:

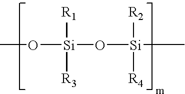

the silicon atoms being able to be substituted by the same or different alkyl radicals and/or aryl radicals which are represented here in generalized form by the radicals R₁-R₄ (in other words the number of different radicals is not necessarily restricted to 4). m can assume values of 2-200,000. Here, aryl preferably stands for phenyl, unless otherwise stated.

Cyclic silicones to be used advantageously according to the invention are generally characterized by structural elements, as follows:

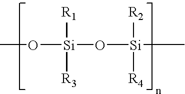

the silicon atoms being able to be substituted by the same or different alkyl radicals and/or aryl radicals which are represented here in generalized form by the radicals R₁-R₄ (in other words the number of different radicals is not necessarily restricted to 4). n can assume values of 3/2 to 20. Fractional values of n take into account that odd numbers of siloxyl groups can be present in the cycle.

Cyclomethicon (e.g. decamethylcyclopentasiloxane) is used advantageously as silicone oil to be used according to the invention. But other silicone oils are also to be used advantageously within the meaning of the present invention, for example undecamethylcyclotrisiloxane, polydimethylsiloxane, poly(methylphenylsiloxane), cetyldimethicon, behenoxydimethicon.

Mixtures of cyclomethicon and isotridecyl isononanoate and also of cyclomethicon and 2-ethylhexyl isostearate are also advantageous.

However it is also advantageous to choose silicone oils of similar constitution as the above-named compounds, the organic side chains of which are derivatized, are for example polyethoxylated and/or polypropoxylated. These include for example polysiloxane-polyalkyl-polyether copolymers such as cetyl-dimethicon-copolyol, (cetyl-dimethicon-copolyol (and) polyglyceryl-4-isostearate (and) hexyl laurate).

Mixtures of cyclomethicon and isotridecyl isononanoate, of cyclomethicon and 2-ethylhexyl isostearate are also particularly advantageous.

The aqueous phase of the preparations according to the invention optionally advantageously contains alcohols, diols or polyols of low C number, and also their ethers, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monethyl ether and analogous products, also alcohols of low C number, e.g. ethanol, isopropanol, 1,2-propanediol, glycerol and also in particular one or more thickening agents which can advantageously be chosen from the group silicone dioxide, aluminium silicates.

Preparations according to the invention present as emulsions preferably contain one or more emulsifiers. These emulsifiers can advantageously be chosen from the group of non-ionic, anionic, cationic or amphoteric emulsifiers.

Non-ionic emulsifiers include (1) partial fatty acid esters and fatty acid esters of polyhydric alcohols and their ethoxylated derivatives (e.g. glyceryl monostearates, sorbitan stearates, glyceryl stearyl citrates, sucrose stearates); (2) ethoxylated fatty alcohols and fatty acids; (3) ethoxylated fatty amines, fatty acid amides, fatty acid alkanol amides; (4) alkylphenol polyglycol ethers (e.g. Triton X).

Anionic emulsifiers include soaps (e.g. sodium stearate); fatty alcohol sulfates; mono-, di- and trialkyl phosphonic acid esters and their ethoxylates.

Cationic emulsifiers include quaternary ammonium compounds with a long-chained aliphatic radical, e.g. distearyl dimonium chloride.

Amphoteric emulsifiers include alkylaminoalkanecarboxylic acids, betaines, sulfobetaines, imidazoline derivatives.

There are also naturally occurring emulsifiers, which include beeswax, wool wax, lecithin and sterols.

O/W emulsifiers can advantageously be chosen for example from the group of polyethoxylated or polypropoxylated or polyethoxylated and polypropoxylated products, e.g. fatty alcohol ethoxylates, ethoxylated wool wax alcohols, polyethylene glycol ethers of general formula R—O—(—CH₂—CH₂—O—)ₙ—R′, fatty acid ethoxylates of the general formula R—COO—(—CH₂—CH₂—O—)ₙ—H, etherified fatty acid ethoxylates of general formula R—COO—(—CH₂—CH₂—O—)ₙ—R′, esterified fatty acid ethoxylates of general formula R—COO—(—CH₂—CH₂—O—)ₙ—C(O)—R′, polyethylene glycol glycerol fatty acid esters, ethoxylated sorbitan esters, cholesterol ethoxylates, ethoxylated triglycerides, alkyl ether carboxylic acids of general formula R—O—(—CH₂—CH₂—O—)ₙ—CH₂—COOH, polyoxyethylene sorbitol fatty acid esters, alkyl ether sulfates of general formula R—O—(—CH₂—CH₂—O—)ₙ—SO₃—H, fatty alcohol propoxylates of general formula R—O—(—CH₂—CH(CH₃)—O—)ₙ—H, polypropylene glycol ethers of general formula R—O—(—CH₂—CH(CH₃)—O—)ₙ—R′, propoxylated wool wax alcohols, etherified fatty acid propoxylates, R—COO—(—CH₂—CH(CH₃)—O—)ₙ—R′, esterified fatty acid propoxylates of general formula R—COO—(—CH₂—CH(CH₃)—O—)ₙ—C(O)—R′, fatty acid propoxylates of general formula R—COO—(—CH₂—CH(CH₃)—O—)ₙ—H, polypropylene glycol glycerol fatty acid esters, propoxylated sorbitan esters, cholesterol propoxylates, propoxylated triglycerides, alkyl ether carboxylic acids of general formula R—O—(—CH₂—CH(CH₃)O—)ₙ—CH₂—COOH, alkyl ether sulfates or acids of general formula R—O—(—CH₂—CH(CH₃)—O—)ₙ—SO₃—H on which these sulfates are based, fatty alcohol ethoxylates/propoxylates of general formula R—O—Xₙ—Yₘ—H, polypropylene glycol ethers of general formula R—O—Xₙ—Yₘ—R′, etherified fatty acid propoxylates of general formula R—COO—Xₙ—Yₘ—R′, fatty acid ethoxylates/propoxylates of general formula R—COO—Xₙ—Yₘ—H.

In all cases the variables n and m each stand, independently of each other, for an integer from 1 to 40, preferably 5 to 30.

Particularly advantageously according to the invention, the polyethoxylated or polypropoxylated or polyethoxylated and polypropoxylated O/W emulsifiers used are chosen from the group of substances with HLB values from 11-18, quite particularly advantageously with HLB values from 14.5-15.5, provided the O/W emulsifiers have saturated radicals R and R′. If the O/W emulsifiers have unsaturated radicals R and/or R′, or if isoalkyl derivatives are present, the preferred HLB value of such emulsifiers can also be lower or higher.

It is advantageous to choose the fatty alcohol ethoxylates from the group of ethoxylated stearyl alcohols, cetyl alcohols, cetyl stearyl alcohols (cetearyl alcohols). Particularly preferred are:

polyethylene glycol (13) stearyl ether (steareth-13), polyethylene glycol (14) stearyl ether (steareth-14), polyethylene glycol (15) stearyl ether (steareth-15), polyethylene glycol (16) stearyl ether (steareth 16), polyethylene glycol (17) stearyl ether (steareth-17), polyethylene glycol (18) stearyl ether (steareth-18), polyethylene glycol (19) stearyl ether (steareth-19), polyethylene glycol (20) stearyl ether (steareth-20),
polyethylene glycol (12) isostearyl ether (isosteareth-12), polyethylene glycol (13) isostearyl ether (isosteareth-13), polyethylene glycol (14) isostearyl ether (isosteareth-14), polyethylene glycol (15) isostearyl ether (isosteareth-15), polyethylene glycol (16) isostearyl ether (isosteareth-16), polyethylene glycol (17) isostearyl ether (isosteareth-17), polyethylene glycol (18) isostearyl ether (isosteareth-18), polyethylene glycol (19) isostearyl ether (isosteareth-19), polyethylene glycol (20) isostearyl ether (isosteareth-20),
polyethylene glycol (13) cetyl ether (ceteth-13), polyethylene glycol (14) cetyl ether (ceteth-14), polyethylene glycol (15) cetyl ether (ceteth-15), polyethylene glycol (16) cetyl ether (ceteth-16), polyethylene glycol (17) cetyl ether (ceteth-17), polyethylene glycol (18) cetyl ether (ceteth-18), polyethylene glycol (19) cetyl ether (ceteth-19), polyethylene glycol (20) cetyl ether (ceteth-20),
polyethylene glycol (13) isocetyl ether (isoceteth-13), polyethylene glycol (14) isocetyl ether (isoceteth-14), polyethylene glycol (15) isocetyl ether (isoceteth-15), polyethylene glycol (16) isocetyl ether (isoceteth-16), polyethylene glycol (17) isocetyl ether (isoceteth-17), polyethylene glycol (18) isocetyl ether (isoceteth-18), polyethylene glycol (19) isocetyl ether (isoceteth-19), polyethylene glycol (20) isocetyl ether (isoceteth-20),
polyethylene glycol (12) oleyl ether (oleth-12), polyethylene glycol (13) oleyl ether (oleth-13), polyethylene glycol (14) oleyl ether (oleth-14), polyethylene glycol (15) oleyl ether (oleth-15),
polyethylene glycol (12) lauryl ether (laureth-12), polyethylene glycol (12) isolauryl ether (isolaureth-12),
polyethylene glycol (13) cetyl stearyl ether (ceteareth-13), polyethylene glycol (14) cetyl stearyl ether (ceteareth-14), polyethylene glycol (15) cetyl stearyl ether (ceteareth-15), polyethylene glycol (16) cetyl stearyl ether (ceteareth-16), polyethylene glycol (17) cetyl stearyl ether (ceteareth-17), polyethylene glycol (18) cetyl stearyl ether (ceteareth-18), polyethylene glycol (19) cetyl stearyl ether (ceteareth-19), polyethylene glycol (20) cetyl stearyl ether (ceteareth-20).

It is furthermore advantageous to choose the fatty acid ethoxylates from the following group:

polyethylene glycol (20) stearate, polyethylene glycol (21) stearate, polyethylene glycol (22) stearate, polyethylene glycol (23) stearate, polyethylene glycol (24) stearate, polyethylene glycol (25) stearate,
polyethylene glycol (12) isostearate, polyethylene glycol (13) isostearate, polyethylene glycol (14) isostearate, polyethylene glycol (15) isostearate, polyethylene glycol (16) isostearate, polyethylene glycol (17) isostearate, polyethylene glycol (18) isostearate, polyethylene glycol (19) isostearate, polyethylene glycol (20) isostearate, polyethylene glycol (21) isostearate, polyethylene glycol (22) isostearate, polyethylene glycol (23) isostearate, polyethylene glycol (24) isostearate, polyethylene glycol (25) isostearate,
polyethylene glycol (12) oleate, polyethylene glycol (13) oleate, polyethylene glycol (14) oleate, polyethylene glycol (15) oleate, polyethylene glycol (16) oleate, polyethylene glycol (17) oleate, polyethylene glycol (18) oleate, polyethylene glycol (19) oleate, polyethylene glycol (20) oleate,

Sodium laureth-11-carboxylate can advantageously be used as ethoxylated alkyl ether carboxylic acid or its salt.

Sodium laureth 1-4 sulfate can advantageously be used as alkyl ether sulfate.

Polyethylene glycol (30) cholesteryl ether can advantageously be used as ethoxylated cholesterol derivative. Polyethylene glycol (25) soya sterol has also proved successful.

The polyethylene glycol (60) evening primrose glycerides can advantageously be used as ethoxylated triglycerides.

It is also advantageous to choose the polyethylene glycol glycerol fatty acid esters from the group polyethylene glycol (20) glyceryl laurate, polyethylene glycol (21) glyceryl laurate, polyethylene glycol (22) glyceryl laurate, polyethylene glycol (23) glyceryl laurate, polyethylene glycol (6) glyceryl caprate/caprinate, polyethylene glycol (20) glyceryl oleate, polyethylene glycol (20) glyceryl isostearate, polyethylene glycol (18) glyceryl oleate/cocoate.

It is likewise favourable to choose the sorbitan esters from the group polyethylene glycol (20) sorbitan monolaurate, polyethylene glycol (20) sorbitan monostearate, polyethylene glycol (20) sorbitan monoisostearate, polyethylene glycol (20) sorbitan monopalmitate, polyethylene glycol (20) sorbitan monooleate.

There can be used as advantageous W/O emulsifiers:

fatty alcohols with 8 to 30 carbon atoms, monoglycerol esters of saturated and/or unsaturated, branched and/or unbranched alkane carboxylic acids of a chain length of 8 to 24, in particular 12-18 C atoms, diglycerol esters of saturated and/or unsaturated, branched and/or unbranched alkane carboxylic acids of a chain length of 8 to 24, in particular 12-18 C atoms, monoglycerol ethers of saturated and/or unsaturated, branched and/or unbranched alcohols of a chain length of 8 to 24, in particular 12-18 C atoms, diglycerol ethers of saturated and/or unsaturated, branched and/or unbranched alcohols of a chain length of 8 to 24, in particular 12-18 C atoms, propylene glycol esters of saturated and/or unsaturated, branched and/or unbranched alkane carboxylic acids of a chain length of 8 to 24, in particular 12-18 C atoms and also sorbitan esters of saturated and/or unsaturated, branched and/or unbranched alkane carboxylic acids of a chain length of 8 to 24, in particular 12-18 C atoms.

Particularly advantageous W/O emulsifiers are glyceryl monostearate, glyceryl monoisostearate, glyceryl monomyristate, glyceryl monooleate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monolaurate, sorbitan monocaprylate, sorbitan monoisooleate, saccharose distearate, cetyl alcohol, stearyl alcohol, arachidyl alcohol, behenyl alcohol, isobehenyl alcohol, selachyl alcohol, chimyl alcohol, polyethylene glycol (2) stearyl ether (steareth-2), glyceryl monolaurate, glyceryl monocaprinate, glyceryl monocaprylate.

Preparations according to the invention present as emulsions also preferably contain one or more hydrocolloids. These hydrocolloids can advantageously be chosen from the group of gums, polysaccharides, cellulose derivatives, layered silicates, polyacrylates and/or other polymers.

Preparations according to the invention present as hydrogels contain one or more hydrocolloids. These hydrocolloids can advantageously be chosen from the above-named group.

Gums include plant or tree saps which harden in air and form resins or extracts from water plants. There can advantageously be chosen from this group within the meaning of the present invention for example gum arabic, carob seed powder, tragacanth, karaya, guar gum, pectin, gellan gum, carrageenan, agar, algins, chondrus, xanthan gum.

Also advantageous is the use of derivatized gums such as e.g. hydroxypropyl guar (Jaguar® HP 8).

Polysaccharides and derivatives include e.g. hyaluronic acid, chitin and chitosan, chondroitin sulfates, starch and starch derivatives.

Cellulose derivatives include e.g. methylcellulose, carboxymethylcellulose, hydroxyethylcellulose, hydroxypropylmethyl cellulose.

Layered silicates include naturally occurring and synthetic aluminas such as e.g. montmorillonite, bentonite, hectorite, laponite, magnesium aluminium silicates such as Veegum®. They can be used as such or in modified form such as e.g. stearalkonium hectorites.

Silica gels can also advantageously be used.

Polyacrylates include e.g. carbopol types from Goodrich (Carbopol 980, 981, 1382, 5984, 2984, EDT 2001 or Pemulen TR2).

Polymers include e.g. polyacrylamides (Seppigel 305), polyvinyl alcohols, PVP, PVP/VA copolymers, polyglycols.

According to a further preferred version the oligoribonucleotides used according to the invention are introduced into aqueous systems or surfactant preparations for cleaning skin and hair.

The cosmetic preparations according to the invention also preferably contain, in addition to the named components, excipients such as they are usually used in such preparations, e.g. preservatives, bactericides, deodorants, antiperspirants, insect repellents, vitamins, anti-foaming agents, dyes, pigments with colouring action, thickening agents, plasticizers, moisturizing and/or moisture-containing substances (moisturizers), or other customary constituents of a cosmetic formulation such as polyols, polymers, foam stabilizers, electrolytes, organic solvents or silicone derivatives, antioxidants and in particular UV absorbers.

Moisturizers are substances or substance mixtures which give cosmetic or dermatological preparations the property, after application to or spreading on the surface of the skin, of reducing the moisture loss from the keratin layer (also called transepidermal water loss (TEWL)) and/or positively influencing the hydration of the keratin layer. Advantageous moisturizers within the meaning of the present invention are for example glycerol, lactic acid, pyrrolidone carboxylic acid and urea. It is furthermore particularly advantageous to use polymeric moisturizers from the group of polysaccharides which are soluble in water and/or swellable in water and/or gellable with the help of water. Particularly advantageous are for example hyaluronic acid and/or a fucose-rich polysaccharide which is filed in the Chemical Abstracts under the registration number 178463-23-5 and can be obtained e.g. under the name Fucogel 1000 from SOLABIA S.A.

When used as a moisturizer, glycerol is preferably used in a quantity of 0.05-30 wt.-%, particularly preferably 1-10%.

The cosmetic compositions can also advantageously contain one or more of the following natural active ingredients or a derivative thereof: alpha-liponic acid, phytoene, D-biotin, coenzyme Q10, alpha glycosyl rutin, carnitine, carnosine, natural and/or synthetic isoflavonoids, creatine, hop or hop-malt extract, taurine. Thus it transpired that active ingredients for positively influencing aging skin, which reduce the formation of wrinkles or else reduce existing wrinkles, such as bioquinones and in particular ubiquinone Q10, soya, creatinine, creatine, liponamide, or promote the restructuring of the connective tissue, such as isoflavone, can be very well used in the formulations according to the invention. It also transpired that the formulations are particularly suitable for combination with active ingredients to support skin functions in the case of dry skin, in particular age-dried skin, such as serinol and osmolytes, e.g. taurine. The incorporation of pigmentation modulators also proved advantageous. Here active ingredients are to be named which reduce the pigmentation of the skin and thus lead to a cosmetically desired brightening of the skin and/or reduce the occurrence of age marks and/or brighten existing age marks (tyrosine sulfate, dioic acid (8-hexadecene-1,16-dicarboxylic acid), liponic acid and liponamide, various liquorice extracts, kojic acid, hydroquinone, arbutin, fruit acids, in particular alpha-hydroxy acids (AHAs), bearberry (Uvae ursi), ursolic acid, ascorbic acid, green tea extracts).

According to a particularly preferred version, the compositions according to the invention contain one or more UV absorbers. Preferred UV absorbers are those which absorb in the region of the UVB and/or UVA rays.

Numerous compounds for protection against UVB radiation are known which are derivatives of 3-benzylidene camphor, 4-aminobenzoic acid, cinnamic acid, salicylic acid, benzophenone and also 2-phenylbenzimidazole. Filters with an absorption maximum in the region of 308 nm are preferred, as the maximum erythemic effectiveness of sunlight lies here.

Advantageous UV-A filter substances within the meaning of the present invention are dibenzoylmethane derivatives, in particular 4-(tert.-butyl)-4′-methoxydibenzoylmethane (CAS no. 70356-09-1) which is sold by Givaudan under the mark Parsol® 1789 and by Merck under the trade name Eusolex® 9020.

The preparations according to the invention advantageously contain substances which absorb UV radiation in the UV-A and/or UV-B region, the overall quantity of filter substances being e.g. 0.1 wt.-% to 30 wt.-%, preferably 0.5 to 20 wt.-%, in particular 1.0 to 15.0 wt.-%, relative to the overall mass of the preparations, in order to provide cosmetic preparations which protect hair or skin against the whole range of ultraviolet radiation. They can also serve as sunscreens for hair or skin.

Further advantageous UV-A filter substances are phenylene-1,4-bis-(2-benzimidazyl)-3,3′-5,5′-tetrasulfonic acid

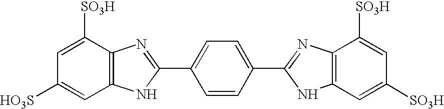

and its salts, in particular the corresponding sodium, potassium or triethanol ammonium salts, in particular phenylene-1,4-bis-(2-benzimidazyl)-3,3′-5,5′-tetrasulfonic acid-bis-sodium salt

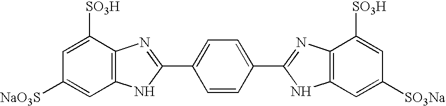

with the INCI name Bisimidazylate which can be obtained for example under the trade name Neo Heliopan AP from Haarmann & Reimer.

Also advantageous are 1,4-di(2-oxo-10-sulfo-3-bornylidenemethyl)-benzene and its salts (in particular the corresponding 10-sulfato compounds, in particular the corresponding sodium, potassium or triethanol ammonium salt), which is also called benzene-1,4-di(2-oxo-3-bornylidenemethyl-10-sulfonic acid) and is characterized by the following structure:

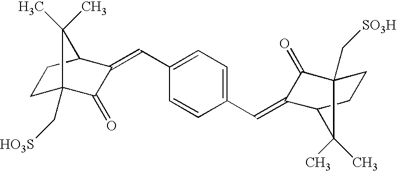

Advantageous UV filter substances within the meaning of the present invention are also so-called broadband filters, i.e. filter substances which absorb both UV-A and UV-B radiation.

Advantageous broadband filters or UV-B filter substances are for example bis-resorcinyl triazine derivatives with the following structure:

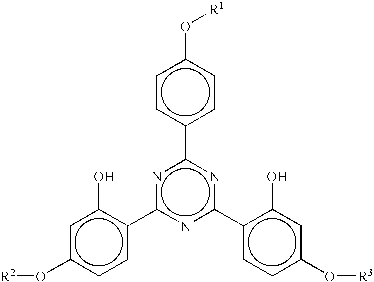

R¹, R² and R³ being chosen independently from one another from the group of branched and unbranched alkyl groups with 1 to 10 carbon atoms or representing a single hydrogen atom. Particularly preferred are 2,4-bis-{[4-(2-ethyl-hexyloxy)-2-hydroxy]-phenyl}-6-(4-methoxyphenyl)-1,3,5-triazine (INCI: Aniso Triazine), which can be obtained under the trade name Tinosorb® S from CIBA-Chemikalien GmbH, and 4,4′-4″-(1,3,5-triazine-2,4,6-triyltriimino)-tris-benzoic acid-tris(2-ethylhexylester), synonym: 2,4,6-tris-[anilino-(p-carbo-2′-ethyl-1′-hexyloxy)]-1,3,5-triazine (INCI: Octyl Triazone), which is sold by BASF Aktiengesellschaft under the trade name UVINUL® T 150.

Other UV filter substances which have the structural unit

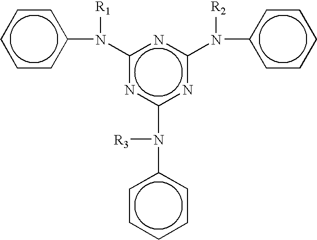

are also advantageous UV filter substances within the meaning of the present invention, for example the s-triazine derivatives described in the European unexamined patent application EP 570 838 A1, the chemical structure of which is represented by the generic formula

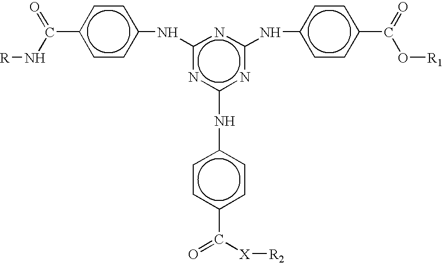

in which
R represents a branched or unbranched C₁-C₁₈ alkyl radical, a C₅-C₁₂ cycloalkyl radical, optionally substituted by one or more C₁-C₄ alkyl groups,
X represents an oxygen atom or an NH group,
R₁ stands for a branched or unbranched C₁-C₁₈ alkyl radical, a C₅-C₁₂ cycloalkyl radical, optionally substituted by one or more C₁-C₄ alkyl groups, or a hydrogen atom, an alkali metal atom, an ammonium group or a group of the formula

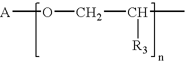

in which
A represents a branched or unbranched C₁-C₁₈ alkyl radical, a C₅-C₁₂ cycloalkyl or aryl radical, optionally substituted by one or more C₁-C₄ alkyl groups,
R₃ represents a hydrogen atom or a methyl group,
n represents a number from 1 to 10,
R₂ stands for a branched or unbranched C₁-C₁₈ alkyl radical, a C₅-C₁₂ cycloalkyl radical, optionally substituted by one or more C₁-C₄ alkyl groups if X represents the NH group, and
   a branched or unbranched C₁-C₁₈ alkyl radical, a C₅-C₁₂ cycloalkyl radical, optionally substituted by one or more C₁-C₄ alkyl groups, or a hydrogen atom, an alkali metal atom, an ammonium group or a group of the formula

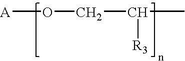

in which
A represents a branched or unbranched C₁-C₁₈ alkyl radical, a C₅-C₁₂ cycloalkyl or aryl radical, optionally substituted by one or more C₁-C₄ alkyl groups,
R₃ represents a hydrogen atom or a methyl group,
n represents a number from 1 to 10,
if X represents an oxygen atom.

A particularly advantageous UV filter substance within the meaning of the present invention is also an unsymmetrically substituted s-triazine the chemical structure of which is reproduced by the formula

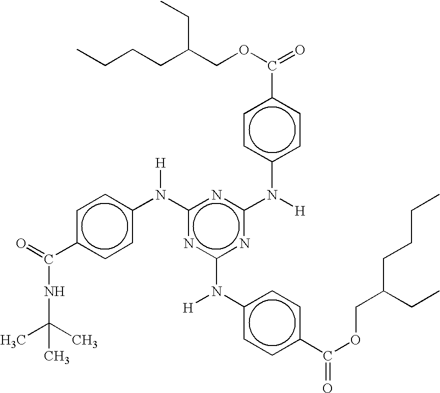

which is also called dioctylbutylamidotriazone (INCI: Dioctylbutamido-Triazone) hereafter and is available under the trade name UVASORB HEB from Sigma 3V.

Bis-resorcinyl triazine derivatives to be used advantageously the chemical structure of which is reproduced by the generic formula

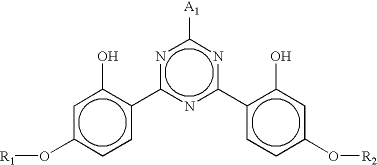

are also described in the European unexamined patent application EP 775 698, R₁, R₂ and A₁ representing very different organic radicals.

Also advantageous within the meaning of the present invention are 2,4-bis-{[4-(3-sulfonato)-2-hydroxypropyloxy)-2-hydroxy]-phenyl}-6-(4-methoxyphenyl)-1,3,5-triazine sodium salt, 2,4-bis-{[4-(3-(2-propyloxy)-2-hydroxy-propyloxy)-2-hydroxy]-phenyl}-6-(4-methoxyphenyl)-1,3,5-triazine, 2,4-bis-{[4-(2-ethylhexyloxy)-2-hydroxy]-phenyl}-6-[4-(2-methoxyethyl-carboxyl)-phenylamino]-1,3,5-triazine, 2,4-bis-{[4-(3-(2-propyloxy)-2-hydroxy-propyloxy)-2-hydroxy]-phenyl}-6-[4-(2-ethyl-carboxyl)-phenylamino]-1,3,5-triazine, 2,4-bis-{[4-(2-ethyl-hexyloxy)-2-hydroxy]-phenyl}-6-(1-methyl-pyrrol-2-yl)-1,3,5-triazine, 2,4-bis-{[4-tris(trimethylsiloxy-silylpropyloxy)-2-hydroxy]-phenyl}-6-(4-methoxyphenyl)-1,3,5-triazine, 2,4-bis-{[4-(2″-methylpropenyloxy)-2-hydroxy]-phenyl}-6-(4-methoxyphenyl)-1,3,5-triazine and 2,4-bis-{[4-(1′,1′,3′,5′,5′,5′-heptamethylsiloxy-2″-methyl-propyloxy)-2-hydroxy]-phenyl-6-(4-methoxyphenyl)-1,3,5-triazine.

An advantageous broadband filter within the meaning of the present invention is 2,2′-methylene-bis-(6-(2H-benzotriazol-2-yl)-4-(1,1,3,3-tetramethylbutyl)-phenol) [INCI: Bisoctyltriazole] which is characterized by the chemical structural formula

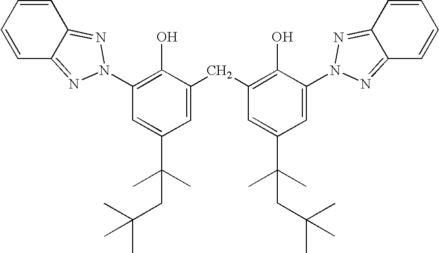

and can be obtained under the trade name Tinosorb® M from CIBA-Chemikalien GmbH.

An advantageous broadband filter within the meaning of the present invention is also 2-(2H-benzotriazol-2-yl)-4-methyl-6-[2-methyl-3-[1,3,3,3-tetramethyl-1-[(trimethylsilyl)oxy]disiloxanyl]propyl]-phenol (CAS no.: 155633-54-8) with the INCI name Drometrizole Trisiloxane, which is characterized by the chemical structural formula

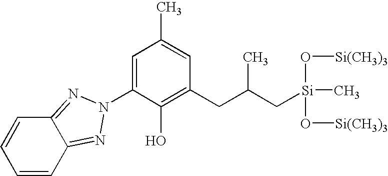

The UVB filters can be oil-soluble or water-soluble. Advantageous oil-soluble UVB filter substances are e.g.: 3-benzylidene camphor derivatives, preferably 3-(4-methylbenzylidene) camphor, 3-benzylidene camphor; 4-aminobenzoic acid derivatives, preferably 4-(dimethylamino)benzoic acid (2-ethylhexyl) ester, 4-(dimethylamino) benzoic acid amyl ester; 2,4,6-trianilino-(p-carbo-2′-ethyl-1′-hexyloxy)-1,3,5-triazine; esters of benzalmalonic acid, preferably 4-methoxybenzalmalonic acid di(2-ethylhexyl) ester; esters of cinnamic acid, preferably 4-methoxycinnamic acid (2-ethylhexyl) ester, 4-methoxycinnamic acid isopentyl ester; derivatives of benzophenone, preferably 2-hydroxy-4-methoxybenzophenone, 2-hydroxy-4-methoxy-4′-methylbenzophenone, 2,2′-dihydroxy-4-methoxybenzophenone and also UV filters bound to polymers.

Advantageous water-soluble UVB filter substances are e.g. salts of 2-phenylbenzimidazole-5-sulfonic acid, such as its sodium, potassium or its triethanol ammonium salt, and also sulfonic acid itself; sulfonic acid derivatives of 3-benzylidene camphor such as e.g. 4-(2-oxo-3-bornylidenemethyl)-benzenesulfonic acid, 2-methyl-5-(2-oxo-3-bornylidenemethyl) sulfonic acid and its salts.

A further light-protection filter substance to be used advantageously according to the invention is ethylhexyl-2-cyano-3,3-diphenylacrylate (octocrylene) which can be obtained from BASF under the name Uvinul® N539 and is characterized by the following structure:

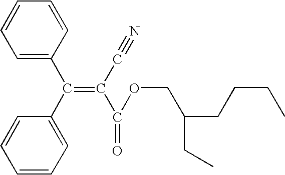

It can also be of considerable advantage to use polymer-bound or polymeric UV filter substances in preparations according to the present invention, in particular those such as are described in WO-A-92/20690.

It can also be advantageous where appropriate according to the invention to incorporate further UV-A and/or UV-B filters into cosmetic or dermatological preparations, for example certain salicylic acid derivatives such as 4-isopropylbenzyl salicylate, 2-ethylhexyl salicylate (=octyl salicylate), homomethyl salicylate.

The list of the named UV filters which can be used within the meaning of the present invention is not of course intended to be limitative.

The compositions according to the invention can also be antioxidants to protect the cosmetic preparation itself or to protect the constituents of the cosmetic preparations against harmful oxidation processes.

The antioxidants are advantageously chosen from the group consisting of amino acids (e.g. glycine, histidine, tyrosine, tryptophan) and their derivatives, imidazoles (e.g. urocanic acid) and their derivatives, peptides such as D,L-carnosine, D-carnosine, L-carnosine and their derivatives (e.g. anserine), carotenoids, carotenes (e.g. α-carotene, β-carotene, lycopine) and their derivatives, aurothioglucose, propylthiouracil and other thiols (e.g. thioredoxin, glutathion, cysteine, cystine, cystamine and its glycosyl, N-acetyl, methyl, ethyl, propyl, amyl, butyl and lauryl, palmitoyl, oleyl, γ-linoleyl, cholesteryl and glyceryl esters) and also their salts, dilauryl thiodipropionate, distearyl thiodipropionate, thiodipropionic acid and its derivatives (esters, ethers, peptides, lipids, nucleotides, nucleosides and salts) and also sulfoximine compounds (e.g. buthionine sulfoximines, homocysteine sulfoximine, buthionine sulfones, penta-, hexa-, heptathionine sulfoximine) in very low compatible dosages (e.g. pmol to μmol/kg), furthermore (metal) chelators (e.g. α-hydroxy fatty acids, palmitic acid, phytinic acid, lactoferrin), α-hydroxy acids (e.g. citric acid, lactic acid, malic acid), humic acid, bile acid, bile extracts, bilirubin, biliverdin, EDTA, EGTA and their derivatives, unsaturated fatty acids and their derivatives (e.g. γ-linoleic acid, linolic acid, oleic acid), folic acid and its derivatives, alanine diacetic acid, flavonoids, polyphenols, catechins, vitamin C and derivatives (e.g. ascorbyl palmitate, Mg ascorbyl phosphate, ascorbyl acetate), tocopherols and derivatives (e.g. vitamin E acetate), and also coniferyl benzoate of gum benzoin, rutinic acid and its derivatives, ferulic acid and its derivatives, butylhydroxytoluene, butylhydroxyanisole, nordihydroguaiac resin acid, nordihydroguaiaretic acid, trihydroxybutyrophenone, uric acid and its derivatives, mannose and its derivatives, zinc and its derivatives (e.g. ZnO, ZnSO₄), selenium and its derivatives (e.g. selenium methionine), stilbenes and their derivatives (e.g. stilbene oxide, transstilbene oxide) and the derivatives suitable according to the invention (salts, esters, ethers, sugars, nucleotides, nucleosides, peptides and lipids) of these named active ingredients.

The quantity of antioxidants (one or more compounds) in the preparations is preferably 0.001 to 30 wt.-%, particularly preferably 0.05-20 wt.-%, in particular 1-10 wt.-% relative to the overall mass of the preparation.

Cosmetic and therapeutic preparations according to the invention also advantageously contain inorganic pigments based on metal oxides and/or other metal compounds poorly soluble or insoluble in water, in particular the oxides of titanium (TiO₂), zinc, (ZnO), iron (e.g. Fe₂O₃), zirconium (ZrO₂), silicon (SiO₂), manganese (e.g. MnO), aluminium (Al₂O₃), cerium (e.g. Ce₂O₃), mixed oxides of the corresponding metals and also mixtures of such oxides. Particularly preferably these are TiO₂-based pigments.

It is particularly advantageous within the meaning of the present invention, although not essential, for the inorganic pigments to be present in hydrophobic form, i.e., for their surface to have been hydrophobically treated. This surface treatment can consist of the pigments being provided with a thin hydrophobic layer using processes known per se.

Such a process consists for example of producing the hydrophobic surface layer according to a reaction as per

nTiO₂+m(RO)₃Si—R′->nTiO₂(surf.).

n and m are stoichiometric parameters to be applied as wished, R and R′ the desired organic radicals. Hydrophobized pigments prepared for example analogously to DE-OS 33 14 742 are advantageous.

Advantageous TiO₂ pigments can be obtained for example under the trade names MT 100 T from TAYCA, also M 160 from Kemira and also T 805 from Degussa.

Preparations according to the invention can also, particularly if crystalline or microcrystalline solids, for example inorganic micropigments, are to be incorporated into the preparations according to the invention, contain anionic, non-ionic and/or amphoteric surfactants. Surfactants are amphiphilic substances which can dissolve organic, non-polar substances in water.

The hydrophilic portions of a surfactant molecule are mostly polar functional groups, for example —COO⁻, —OSO₃²⁻, —SO₃⁻, whereas the hydrophobic parts are as a rule non-polar hydrocarbon radicals. Surfactants are generally classified according to the type and charge of the hydrophilic molecule part. A distinction can be made between four groups, namely anionic surfactants, cationic surfactants, amphoteric surfactants and non-ionic surfactants.

As functional groups, anionic surfactants usually have carboxylate, sulfate or sulfonate groups. In aqueous solution, they form negatively charged organic ions in an acid or neutral environment. Cationic surfactants are almost exclusively characterized by the presence of a quaternary ammonium group. In aqueous solution, they form positively charged organic ions in an acid or neutral environment. Amphoteric surfactants contain both anionic and cationic groups and accordingly behave as anionic or cationic surfactants in aqueous solution, depending on the pH value. They have a positive charge in a strongly acid environment and a negative charge in an alkaline environment. In the neutral pH range, on the other hand, they are zwitterionic, as the following example is intended to illustrate:

pH=2 RNH₂+CH₂CH₂COOH X⁻
(X⁻=any anion, e.g. Cl⁻)
   pH=7 RNH₂⁺CH₂CH₂COO⁻
   pH=12 RNHCH₂CH₂COO⁻ B⁺
(B⁺=any cation, e.g. Na⁺)

Polyether chains are typical of non-ionic surfactants. Non-ionic surfactants do not form ions in an aqueous medium.

Anionic surfactants to be used advantageously are:

acylamino acids (and their salts), such as (1) acyl glutamates, for example sodium acyl glutamate, Di-TEA-palmitoyl aspartate and sodium caprylic/capric glutamate; (2) acyl peptides, for example palmitoyl-hydrolyzed lactoprotein, sodium cocoyl-hydrolyzed soya protein and sodium/potassium cocoyl-hydrolyzed collagen; (3) sarcosinates, for example myristoyl sarcosine, TEA-lauroyl sarcosinate, sodium lauroyl sarcosinate and sodium cocoyl sarcosinate; (4) taurates, for example sodium lauroyl taurate and sodium methyl cocoyl taurate; (5) acyl lactylates, such as lauroyl lactylate and caproyl lactylate; (6) alaninates;
carboxylic acids and derivatives, such as for example lauric acid, aluminium stearate, magnesium alkanolate and zinc undecylenate; ester carboxylic acids, for example calcium stearoyl lactylate, laureth-6 citrate and sodium PEG-4 lauramide carboxylate; ether carboxylic acids, for example sodium laureth-13 carboxylate and sodium PEG-6 cocamide carboxylate;
carboxylic acids, ester carboxylic acids and ether carboxylic acids preferably contain 1 to 50 and in particular 2 to 30 carbon atoms.
phosphoric acid esters and salts, such as for example DEA-oleth-10-phosphate and dilaureth-4 phosphate;
sulfonic acids and salts, such as (1) acyl isethionates, e.g. sodium/ammonium cocoyl isethionate; (2) alkylaryl sulfonates; (3) alkyl sulfonates, for example sodium cocomonoglyceride sulfate, sodium C₁₂₋₁₄ olefin sulfonate, sodium lauryl sulfoacetate and magnesium PEG-3 cocamide sulfate; (4) sulfosuccinates, for example dioctyl sodium sulfosuccinate, disodium laureth sulfosuccinate, disodium lauryl sulfosuccinate and disodium undecyleneamido-MEA sulfosuccinate;
sulfuric acid esters, such as (1) alkyl ether sulfate, for example sodium, ammonium, magnesium, MIPA, TIPA laureth sulfate, sodium myreth sulfate and sodium C₁₂₋₁₃ pareth sulfate; (2) alkyl sulfates, for example sodium, ammonium and TEA lauryl sulfate.

Cationic surfactants to be used advantageously are alkylamines, alkylimidazoles, ethoxylated amines and quaternary surfactants and also esterquats.

Quaternary surfactants contain at least one N atom which is covalently bound to 4 alkyl or aryl groups. This leads, irrespective of the pH value, to a positive charge. Alkylbetaine, alkylamidopropylbetaine and alkylamidopropylhydroxysulfaine are advantageous. The cationic surfactants used according to the invention can also preferably be chosen from the group of quaternary ammonium compounds, in particular benzyltrialkyl ammonium chlorides or bromides, such as for example benzyldimethylstearyl ammonium chloride, also alkyltrialkyl ammonium salts, for example cetyltrimethyl ammonium chloride or bromide, alkyldimethylhydroxyethyl ammonium chlorides or bromides, dialkyldimethyl ammonium chlorides or bromides, alkylamide ethyltrimethyl ammonium ether sulfates, alkylpyridinium salts, for example lauryl or cetylpyrimidinium chloride, imidazoline derivatives and compounds with a cationic character such as amine oxides, for example alkyldimethylamine oxides or alkylaminoethyldimethylamine oxides. Cetyltrimethyl ammonium salts in particular are advantageously to be used.

Amphoteric surfactants to be used advantageously are (1) acyl/dialkylethylenediamine, for example sodium acylamphoacetate, disodium acylamphodipropionate, disodium alkylamphodiacetate, sodium acylamphohydroxypropylsulfonate, disodium acylamphodiacetate and sodium acylamphopropionate; (2) N-alkylamino acids, for example aminopropylalkyl glutamide, alkylamino propionic acid, sodium alkylimidodipropionate and lauroamphocarboxyglycinate.

Non-ionic surfactants to be used advantageously are (1) alcohols; (2) alkanol amides, such as MEA/DEA/MIPA cocamides; (3) amine oxides, such as cocoamidopropylamine oxide; (4) esters which form through esterification of carboxylic acids with ethylene oxide, glycerol, sorbitan or other alcohols; (5) ethers, for example ethoxylated/propoxylated alcohols, ethoxylated/propoxylated esters, ethoxylated/propoxylated glycerol esters, ethoxylated/propoxylated cholesterols, ethoxylated/propoxylated triglyceride esters, ethoxylated/propoxylated lanolin, ethoxylated/propoxylated polysiloxanes, propoxylated POE ethers and alkylpolyglycosides such as lauryl glucoside, decyl glycoside and coco glycoside; (6) sucrose esters, ethers; (7) polyglycerol esters, diglycerol esters, monoglycerol esters; (8) methyl glucose esters, esters of hydroxy acids.

The use of a combination of anionic and/or amphoteric surfactants with one or more non-ionic surfactants is also advantageous.

The surfactant can be present in the preparations according to the invention in a concentration between 1 and 95 wt.-%, relative to the overall mass of the preparations.

Preparations for medical application are no different in their composition from the cosmetic products and can likewise contain the abovenamed substances. They differ from the latter primarily in that they must undergo a special approval procedure.

The invention is explained in more detail below using embodiments. All the numerical values in the examples relate to wt.-% unless otherwise stated.

### EXAMPLES

### Example 1

### Measurement of the Inhibition of MMP1-Expression in HeLaS3 Cells by dsRNA

In order to ascertain the effectiveness of oligonucleotides according to the invention tumoral cells of the HeLaS3 line were used. The expression of the metalloproteinase MMP-1 by the cells does not occur endogenously but only under “cell stress” and was induced by UVA irradiation and addition of phorbol-12-myristat-13-acetate (TPA).

For this purpose, on the day before the induction, the cells (in HAM's F12 medium with 10% foetal calf serum) were seeded at a density of 0.5×10⁵ cells per well (24 wells per plate). Induction then took place through UVA irradiation at an intensity of 15 J/cm² with addition of TPA (150 ng/ml). The cells were then washed twice with physiological, phosphate-buffered saline; PBS (−/−) and covered with fresh HAM's F12 medium (Gibco). In order to expose the cells to a further stress factor, medium with a lower concentration of foetal calf serum (FCS, 0.2% instead of 10%, Gibco) was used. 24 hours after the induction, transfection took place. It was carried out using a mixture of cationic lipids (N-[1-(2,3-dioleoyloxy)propyl]-n,n,n-trimethylammonium chloride and dioleoylphosphatidyl-ethanolamine; Lipofektamin Plus reagent, Gibco). Some of the cells were co-transfected with 0.4 μg pEGFP plasmid and 0.21 μg anti-MMP-1 dsRNA (dsRNA obtained by hybridizing the sequences SEQ ID Nos. 18 and 19, the dsRNA has two overhanging dT residues at the 3′ position in each case) analogously to Elbashir et al. Nature 411 (2001) 494-498. More cells were transfected with the plasmid only. The plasmid pEGFP contains a coding sequence for “Green Fluorescence Protein”, which as section of a peptide chain contains a chromophore. The protein has the property of fluorescing intensively green under UV light and can thus be used as a direct transfection control under the microscope. Samples not transfected with plasmid or dsRNA served as comparison. 3 samples were taken for each formulation.

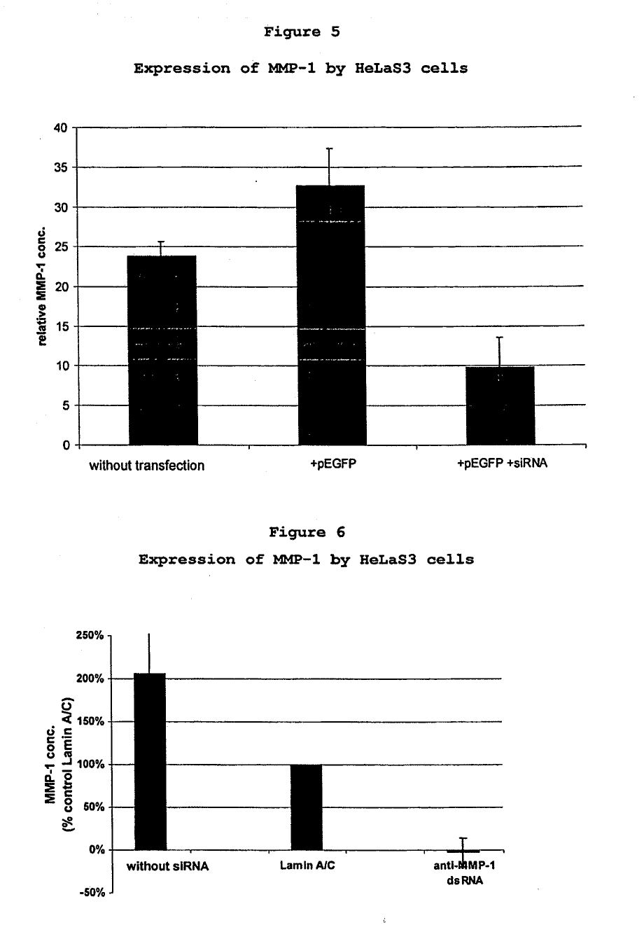

After microscopic examination of the cells the MMP-1 content in the cell supernatant was measured by means of ELISA (MMP-1 ELISA; Oncogen). The results found are represented graphically in FIG. 5. The relative concentration, standardized via the protein content, of metalloproteinase MMP-1 in the cell supernatant can be seen here for each experiment. The averages from 3 measurements are shown in each case. Transfection of the cells with pEGFP alone effects an increase in MMP-1 formation, which is attributable to an activation of the MMP-1 by the transfections, which means a stress situation for the cells. Transfection with pEGFP and anti-MMP-1-dsRNA leads to a drop of approximately 60% in the MMP-1 concentration compared with the non-transfected cells and approximately 70% compared with the pEGFP-transfected cells.

### Example 2

### Preparation of Pit Emulsions

By mixing the components given in the table, phase inversion temperature emulsions (PIT emulsions) of the composition which is likewise given were prepared. dsRNA which was obtained by hybridizing the sequences SEQ ID Nos. 12 and 13 was used as oligoribonucleotide. The dsRNA has two dT overhangs at the 3′ position in each case. The dsRNA is specific to the cDNA of the MMP-1 and inhibits the expression of the gene of this enzyme by RNA interference. It is therefore called anti-MMP-1 dsRNA. The other abbreviations used in the examples are to be understood accordingly.

TABLE 1
PIT Emulsions

| Emulsion No. | 1 | 2 | 3 | 4 | 5 |
|---|---|---|---|---|---|
| Self-emulsifying glycerol monostearate | 0.50 | | 3.00 | 2.00 | 4.00 |
| Polyoxyethylene (12) cetyl stearyl ether | | 5.00 | | 1.00 | 1.50 |
| Polyoxyethylene (20) cetyl stearyl ether | | | | 2.00 | |
| Polyoxyethylene (30) cetyl stearyl ether | 5.00 | | 1.00 | | |
| Stearyl alcohol | | | 3.00 | | 0.50 |
| Cetyl alcohol | 2.50 | 1.00 | | 1.50 | |
| 2-ethylhexyl methoxycinnamate | | | | 5.00 | 8.00 |
| 2,4-bis-(4-(2-ethyl-hexyloxy)2-hydroxyl)- | | 1.50 | | 2.00 | 2.50 |
| phenyl)-6-(4-methoxyphenyl)-1,3,5)- | | | | | |
| triazine | | | | | |
| 1-(4-tert-butylphenyl)-3-(4- | | | 2.00 | | |
| methoxyphenyl)-1,3-propanedione | | | | | |
| Diethylhexylbutamido-triazone | 1.00 | 2.00 | | 2.00 | |
| Ethylhexyltriazone | 4.00 | | 3.00 | 4.00 | |
| 4-methylbenzylidene camphor | | 4.00 | | | 2.00 |
| Octocrylene | | 4.00 | | | 2.50 |
| Phenylene-1,4-bis-(monosodium, 2- | | | 0.50 | | 1.50 |
| benzimidazyl-5,7-disulfonic acid) | | | | | |
| Phenylbenzimidazole sulfonic acid | 0.50 | | | 3.00 | |
| C12-15 alkyl benzoate | | 2.50 | | | 5.00 |
| Titanium dioxide | 0.50 | 1.00 | | 3.00 | 2.00 |
| Zinc oxide | 2.00 | | 3.00 | 0.50 | 1.00 |
| Dicaprylyl ether | | | 3.50 | | |
| Butylene glycol-dicaprylate/-dicaprate | 5.00 | | | 6.00 | |
| Dicaprylyl carbonate | | | 6.00 | | 2.00 |
| Dimethicon polydimethylsiloxane | | 0.50 | 1.00 | | |
| Phenylmethylpolysiloxane | 2.00 | | | 0.50 | 0.50 |
| Shea butter | | 2.00 | | | 0.50 |
| PVP hexadecene copolymer | 0.50 | | | 0.50 | 1.00 |
| Glycerol | 3.00 | 7.50 | 5.00 | 7.50 | 2.50 |
| Tocopherol acetate | 0.50 | | 0.25 | | 1.00 |
| anti-MMP1-dsRNA (dsRNA from SEQ | 0.10 | 0.10 | | 0.10 | 0.10 |
| ID Nos. 12 and 13) | | | | | |
| Preservative | q.s | q.s | q.s | q.s | q.s |
| Ethanol | 3.00 | 2.00 | 1.50 | | 1.00 |
| Perfume | q.s | q.s | q.s | q.s | q.s |
| Water | ad. 100 | ad. 100 | ad. 100 | ad. 100 | ad. 100 |

In analogous manner, a PIT emulsion was prepared using dsRNA which was obtained by hybridizing the sequences SEQ Nos. 30 and 31.

### Example 3

### Preparation of Creams Based on Oil-in-Water Emulsions

By mixing the components given in the table, creams the composition of which is likewise given were prepared.

TABLE 2
O/W Creams

| Cream No. | 1 | 2 | 3 | 4 | 5 |
|---|---|---|---|---|---|
| Glyceryl stearate citrate | | | 2.00 | | 2.00 |
| Self-emulsifying glyceryl stearate | 4.00 | 3.00 | | | |
| PEG-40 stearate | 1.00 | | | | |
| Polyglyceryl-3-methylglucose- | | | | 3.00 | |
| distearate | | | | | |
| Sorbitan stearate | | | | | 2.00 |
| Stearic acid | | 1.00 | | | |
| Stearyl alcohol | | | 5.00 | | |
| Cetyl alcohol | 3.00 | 2.00 | | 3.00 | |
| Cetyl stearyl alcohol | | | | | 2.00 |
| Caprylic/capric triglyceride | 5.00 | 3.00 | 4.00 | 3.00 | 3.00 |
| Octyl dodecanol | | | 2.00 | | 2.00 |
| Dicaprylyl ether | | 4.00 | | 2.00 | 1.00 |
| Liquid paraffin | 5.00 | 2.00 | | 3.00 | |
| Titanium dioxide | | | 1.00 | | |
| 4-methylbenzylidene camphor | | | 1.00 | | |
| 1,4-tert-butylphenyl)-3-(4- | | | 0.50 | | |
| methoxyphenyl)-1,3-propanedione | | | | | |
| anti-MMP1-dsRNA (dsRNA from | 0.10 | 0.10 | 0.10 | 0.10 | 0.10 |
| SEQ ID Nos. 12 and 13) | | | | | |
| Tocopherol | 0.1 | | | | 0.20 |
| Biotin | | | 0.05 | | |
| Ethylenediaminetetraacetic acid | 0.1 | | 0.10 | 0.1 | |
| trisodium | | | | | |
| Preservative | q.s | q.s | q.s | q.s | q.s |
| Polyacrylic acid | 3.00 | 0.1 | | 0.1 | 0.1 |
| Caustic soda 45% | q.s | q.s | q.s | q.s | q.s |
| Glycerol | 5.00 | 3.00 | 4.00 | 3.00 | 3.00 |
| Butylene glycol | | 3.00 | | | |
| Perfume | q.s | q.s | q.s | q.s | q.s |
| Water | ad. 100 | ad. 100 | ad. 100 | ad. 100 | ad. 100 |
| Glyceryl stearate citrate | | 2.00 | 2.00 | | |
| Self-emulsifying glyceryl stearate | 5.00 | | | | |
| Stearic acid | | | | 2.50 | 3.50 |
| Stearyl alcohol | 2.00 | | | | |
| Cetyl alcohol | | | | 3.00 | 4.50 |
| Cetyl stearyl alcohol | | 3.00 | 1.00 | | 0.50 |
| C12-15 alkyl benzoate | | 2.00 | 3.00 | | |
| Caprylic/capric triglyceride | 2.00 | | | | |
| Octyl dodecanol | 2.00 | 2.00 | | 4.00 | 6.00 |
| Dicaprylyl ether | | | | | |
| Liquid paraffin | | 4.00 | 2.00 | | |
| Cyclic dimethylpolysiloxane | | | | 0.50 | 2.00 |
| Dimethicon polydimethylsiloxane | 2.00 | | | | |
| Titanium dioxide | 2.00 | | | | |
| 4-methylbenzylidene camphor | 1.00 | | | | 1.00 |
| 1-(4-tert-butylphenyl)-3-(4- | 0.50 | | | | 0.50 |
| methoxyphenyl)-1,3-propanedione | | | | | |
| anti-MMP1-dsRNA (dsRNA from | 0.10 | 0.10 | 0.10 | 0.10 | 0.10 |
| SEQ ID Nos. 12 and 13) | | | | | |
| Tocopherol | | | | | 0.05 |
| Ethylenediaminetetraacetic acid | | | 0.20 | | 0.20 |
| trisodium | | | | | |
| Preservative | q.s | q.s | q.s | q.s | q.s |
| Xanthan gum | | | 0.20 | | |
| Polyacrylic acid | 0.15 | 0.1 | | 0.05 | 0.05 |
| Caustic soda 45% | q.s | q.s | q.s | q.s | q.s |
| Glycerol | 3.00 | | 3.00 | 5.00 | 3.00 |
| Butylene glycol | | 3.00 | | | |
| Ethanol | | 3.00 | | 3.00 | |
| Perfume | q.s | q.s | q.s | q.s | q.s |
| Water | ad. 100 | ad. 100 | ad. 100 | ad. 100 | ad. 100 |

In analogous manner, a cream was prepared using dsRNA which was obtained by hybridizing the sequences SEQ Nos. 30 and 31.

### Example 4

### Preparation of Water-in-Oil Emulsions

By mixing the components given in the table, water-in-oil emulsions, the composition of which is also given, were prepared. dsRNA which was obtained by hybridizing the sense RNA and antisense RNA strands to SEQ ID No. 60 was used as oligoribonucleotide. SEQ ID No. 60 is a section of the cDNA of elastase 2. Both strands of the dsRNA have two 2-deoxythymidine overhangs at the 3′ position in each case.

TABLE 3
W/O Emulsions

| Emulsion No. | 1 | 2 | 3 | 4 | 5 |
|---|---|---|---|---|---|
| Cetyldimethicon copolyol | | 2.50 | | 4.00 | |
| Polyglyceryl-2- | 5.00 | | | | 4.50 |
| dipolyhydroxystearate | | | | | |
| PEG-30-dipolyhydroxystearate | | | 5.00 | | |
| 2-ethylhexyl methoxycinnamate | | 8.00 | | 5.00 | 4.00 |
| 2,4-bis-(4-(2-ethyl-hexyloxy)-2- | 2.00 | 2.50 | | 2.00 | 2.50 |
| hydroxyl)-phenyl)-6-(4- | | | | | |
| methoxyphenyl)-(1,3,5)-triazine | | | | | |
| 1-(4-tert-butylphenyl)-3-(4- | | | 2.00 | 1.00 | |
| methoxyphenyl)-1,3-propanedione | | | | | |
| Diethylhexylbutamido-triazone | 3.00 | 1.00 | | | 3.00 |
| Ethylhexyl triazone | | | 3.00 | 4.00 | |
| 4-methylbenzylidene camphor | | 2.00 | | 4.00 | 2.00 |
| Octocrylene | 7.00 | 2.50 | 4.00 | | 2.50 |
| Diethylhexylbutamido-triazone | 1.00 | | | 2.00 | |
| Phenylene-1-4-bis-(monosodium,2- | 1.00 | 2.00 | 0.50 | | |
| benzimidazyl-5,7-disulfonic acid) | | | | | |
| Phenylbenzimidazole sulfonic acid | 0.50 | | | 3.00 | 2.00 |
| Titanium dioxide | | 2.00 | 1.50 | | 3.00 |
| Zinc oxide | 3.00 | 1.00 | 2.00 | 0.50 | |
| Liquid paraffin | | | 10.0 | | 8.00 |
| C12-15 alkyl benzoate | | | | 9.00 | |
| Dicaprylyl ether | 10.00 | | | | 7.00 |
| Butylene-glycol-dicaprylate/- | | | 2.00 | 8.00 | 4.00 |
| dicaprate | | | | | |
| Dicaprylyl carbonate | 5.00 | | 6.00 | | |
| Dimethicon polydimethylsiloxane | | 4.00 | 1.00 | 5.00 | |
| Phenylmethylpolysiloxane | 2.00 | 25.00 | | | 2.00 |
| Shea butter | | | 3.00 | | |
| PVP hexadecene copolymer | 0.50 | | | 0.50 | 1.00 |
| Octoxyglycerol | | 0.30 | 1.00 | | 0.50 |
| Glycerol | 3.00 | 7.50 | | 7.50 | 2.50 |
| Glycine soya | | 1.00 | 1.50 | | |
| Magnesium sulfate | 1.00 | 0.50 | | 0.50 | |
| Magnesium chloride | | | 1.00 | | 0.70 |
| Tocopherol acetate | 0.50 | | 0.25 | | 1.00 |
| anti-elastase dsRNA | 0.10 | 0.10 | 0.10 | 0.10 | 0.10 |
| Preservative | q.s | q.s | q.s | q.s | q.s |
| Ethanol | 3.00 | | 1.50 | | 1.00 |
| Perfume | q.s | q.s | q.s | q.s | q.s |
| Water | ad. 100 | ad. 100 | ad. 100 | ad. 100 | ad. 100 |

| Emulsion No. | 6 | 7 |
|---|---|---|
| Cetyldimethicon copolyol | | |
| Polyglyceryl-2-dipolyhydroxystearate | 4.00 | 5.00 |
| PEG-30-dipolyhydroxystearate | | |
| Lanolin alcohol | 0.50 | 1.50 |
| Isohexadecane | 1.00 | 2.00 |
| Myristyl-myristate | 0.50 | 1.50 |
| Vaseline | 1.00 | 2.00 |
| 1-(4-tert-butylphenyl)-3-(4- | 0.50 | 1.50 |
| methoxyphenyl)-1,3-propanedione | | |
| 4-methylbenzylidene camphor | 1.00 | 3.00 |
| Butylene-glycol-dicaprylate/-dicaprate | 4.00 | 5.00 |
| Shea butter | | 0.50 |
| Butylene glycol | | 6.00 |
| Octoxyglycerol | | 3.00 |
| Glycerol | 5.00 | |
| Tocopherol acetate | 0.50 | 1.00 |
| anti-elastase dsRNA | 0.10 | 0.10 |
| Trisodium EDTA | 0.20 | 0.20 |
| Preservative | q.s | q.s |
| Ethanol | | 3.00 |
| Perfume | q.s | q.s |
| Water | ad. 100 | ad. 100 |

### Example 5

### Preparation of Hydrodispersions

By mixing the components given in the table, hydrodispersions, the composition of which is also given, were prepared. dsRNA which was obtained by hybridizing the sense RNA and antisense RNA strands to SEQ ID No. 62 was used as oligoribonucleotide. SEQ ID No. 62 is a section of the cDNA of hyaluronidase 2. Both strands of the dsRNA had two 2-deoxythymidine overhangs at the 3′ position in each case.

TABLE 4
Hydrodispersions

| Dispersion No. | 1 | 2 | 3 | 4 | 5 |
|---|---|---|---|---|---|
| Polyoxyethylene (20) cetyl stearyl ether | 1.00 | | | 0.5 | |
| Cetyl alcohol | | | 1.00 | | |
| Sodium polyacrylate | | 0.20 | | 0.30 | |
| Acrylates/C 10-30 alkyl-acrylate cross- | 0.50 | | 0.40 | 0.10 | 0.10 |
| polymer | | | | | |
| Xanthan gum | | 0.30 | 0.15 | | 0.50 |
| 2-ethylhexyl methoxycinnamate | | | | 5.00 | 8.00 |
| 2,4-bis-(4-(2-ethyl-hexyloxy-)2- | | 1.50 | | 2.00 | 2.50 |
| hydroxyl)-phenyl)-6-(4- | | | | | |
| methoxyphenyl)-(1,3,5)-triazine | | | | | |
| 1-(4-tert-butylphenyl)-3-(4- | 1.00 | | 2.00 | | |
| methoxyphenyl)-1,3-propanedione | | | | | |
| Diethylhexylbutamido-triazone | | 2.00 | | 2.00 | 1.00 |
| Ethylhexyl triazone | 4.00 | | 3.00 | 4.00 | |
| 4-methylbenzylidene camphor | 4.00 | 4.00 | | | 2.00 |
| Octocrylene | | 4.00 | 4.00 | | 2.50 |
| Phenylene-1,4-bis-(monosodium, 2- | 1.00 | | 0.50 | | 2.00 |
| benzimidazyl-5,7-disulfonic acid | | | | | |
| Phenylbenzimidazole sulfonic acid | 0.50 | | | 3.00 | |
| Titanium dioxide | 0.50 | | 2.00 | 3.00 | 1.00 |
| Zinc oxide | 0.50 | 1.00 | 3.00 | | 2.00 |
| C12-15 alkyl benzoate | 2.00 | 2.50 | | | |
| Dicaprylyl ether | | 4.00 | | | |
| Butylene glycol-dicaprylate/-dicaprate | 4.00 | | 2.00 | 6.00 | |
| Dicaprylyl carbonate | | 2.00 | 6.00 | | |
| Dimethicon polydimethylsiloxane | | 0.50 | 1.00 | | |
| Phenylmethylpolysiloxane | 2.00 | | | 0.50 | 2.00 |
| Shea butter | | 2.00 | | | |
| PVP hexadecene copolymer | 0.50 | | | 0.50 | 1.00 |
| Octoxyglycerol | | | 1.00 | | 0.50 |
| Glycerol | 3.00 | 7.50 | | 7.50 | 2.50 |
| Glycine soya | | | 1.50 | | |
| Tocopherol acetate | 0.50 | | 0.25 | | 1.00 |
| anti-hyaluronidase dsRNA | 0.10 | 0.10 | 0.10 | 0.10 | 0.10 |
| Preservative | q.s | q.s | q.s | q.s | q.s |
| Ethanol | 3.00 | 2.00 | 1.50 | | 1.00 |
| Perfume | q.s | q.s | q.s | q.s | q.s |
| Water | ad. 100 | ad. 100 | ad. 100 | ad. 100 | ad. 100 |

### Example 6

### Preparation of a Gel Cream

By mixing the components given in the table, a gel cream, the composition of which is also given, was prepared. The pH value of the gel cream was then set to 6.0.

TABLE 5
Gel cream

| | |
|---|---|
| Acrylate/C10-30 alkylacrylate cross-polymer | 0.40 |
| Polyacrylic acid | 0.20 |
| Xanthan gum | 0.10 |
| Cetearyl alcohol | 3.00 |
| C12-15 alkyl benzoate | 4.00 |
| Caprylic/capric triglyceride | 3.00 |
| Cyclic dimethylpolysiloxane | 5.00 |
| anti-MMP1 dsRNA (dsRNA from SEQ ID | 0.10 |
| Nos. 12 and 13) | |
| Glycerol | 3.00 |
| Sodium hydroxide | q.s |
| Preservative | q.s |
| Perfume | q.s |
| Water | ad. 100.0 |
| pH value set to 6.0 | |

In analogous manner, a gel cream was prepared using dsRNA which was obtained by hybridizing the sequences SEQ Nos. 30 and 31.

### Example 7

### Preparation of a Cream on the Basis of a Water-in-Oil Emulsion

By mixing the components given in the table, a cream, the composition of which is also given, was prepared on the basis of a water-in-oil dispersion.

TABLE 6
W/O Cream

| | |
|---|---|
| Polyglyceryl-3-diisostearate | 3.50 |
| Glycerol | 3.00 |
| Polyglyceryl-2-dipolyhydroxystearate | 3.50 |
| anti-MMP1-dsRNA (dsRNA from SEQ ID | 0.10 |
| Nos. 12 and 13) | |
| Preservative | q.s. |
| Perfume | q.s |
| Water | ad. 100.0 |
| Magnesium sulfate | 0.6 |
| Isopropyl stearate | 2.0 |
| Caprylyl ether | 8.0 |
| Cetearyl isononanoate | 6.0 |

In analogous manner, an emulsion was prepared using dsRNA which was obtained by hybridizing the sequences SEQ Nos. 30 and 31.

### Example 8

### Preparation of a Cream on the Basis of a Water-in-Oil-in-Water Emulsion

By mixing the components given in the table, a cream, the composition of which is also given, was prepared on the basis of a water-in-oil-in-water dispersion. dsRNA which was obtained by hybridizing the sense RNA and antisense RNA strands to SEQ ID No. 60 was used as oligoribonucleotide. Both strands of the dsRNA had two 2-deoxythymidine overhangs at the 3′ position in each case.

TABLE 7
W/O/W Cream

| | |
|---|---|
| Glyceryl stearate | 3.00 |
| PEG-100 stearate | 0.75 |
| Behenyl alcohol | 2.00 |
| Caprylic/capric triglyceride | 8.0 |
| Octyl dodecanol | 5.00 |
| C12-15 alkyl benzoate | 3.00 |
| anti-elastase dsRNA | 0.10 |
| Magnesium sulfate (MgSO4) | 0.80 |
| Ethylenediaminetetraacetic acid | 0.10 |
| Preservative | q.s |
| Perfume | q.s |
| Water | ad. 100.0 |
| pH value set to 6.0 | |

### Example 9

### Measurement of the Inhibition of MMP1-Expression in HeLaS3 Cells by dsRNA

In the manner described in Example 1 HeLaS3 cells were transfected with anti-MMP-1 dsRNA based on SEQ NO 33. The dsRNA used had two A overhangs at the 5′ position in addition to the nucleotides of SEQ NO 33 and was supplemented by the complementary strand to the double strand. The concentration of the enzyme MMP-1 in the cell supernatant was then measured by means of ELISA (MMP-1 ELISA, Oncogene). 1×10⁴ cells per well of a 24-well cell culture plate were used. There served as control, on the one hand cells which were not transfected with dsRNA, on the other hand cells which were transfected with anti-lamin A/C dsRNA. Lamin A/C is among the intermediate filaments which form the nuclear lamina. As it can therefore be detected in all eukaryotic cells it is often used as a standard of gene and protein expression experiments. Commercially available “presynthesized” dsRNA lamin A/C Duplices were used (MWG Biotech AG, Ebersberg, Germany). The inhibition of the lamin A/C expression has no influence on the MMP-1 expression. The MMP-1 concentration of the control formulation was defined as 100%. The results are shown in FIG. 6. In each case two measurements were carried out per dsRNA and the results averaged. FIG. 6 shows that the expression of the enzyme MMP-1 was practically completely inhibited by the dsRNA according to the invention.

### Example 10

### Measurement of the Inhibition of MMP1-Expression in Primary Fibroblasts (Female) by dsRNA

In order to carry out the tests fibroblasts isolated from skin biopsy material of a 57-year-old female donor (57w) were used. Fibroblasts are pregenitors of conjunctive tissue cells (fibrocytes).

For the transfection the fibroblasts were seeded at a density of 2×10⁴ cells per well of a cell culture multi-well plate (24 wells per plate) and cultured for 24 hours in complete medium (Dulbecco's modified Eagle's Medium (DMEM, supplier: Gibco Invitrogen/standard cell culture medium); +10% foetal calf serum (FCS, PAA Laboratories, Linz)+1% Glutamax (PAA Laboratories, Linz)+1% penicillin/streptavidin (Pen/Strep; Gibco Invitrogen).

For the transfection of the cells cationic lipids were used (Oligofektamin; Invitrogen). For the transfection formulation (per well) 0.21 μg of the anti-MMP-1 dsRNA described in Example 9 was first dissolved in 40 μl of medium (DMEM without FCS supplementation). Separately from this 1 μl of Oligofektamin (undiluted reagent) was dissolved in 6.5 μl of medium (DMEM without FCS supplementation) and incubated for 5 to 10 minutes at room temperature. After this incubation period the Oligofektamin reagent was added to the dsRNA and the formulations were incubated for another 15 to 20 minutes.

The test procedure and the quantity of dsRNA duplices used were as described in Elbashir et al., in Nature 2001, 411, pages 494-498. A nonsense dsRNA treated in the same manner served as control. This was obtained starting from the sequence of the above active anti-MMP-1 dsRNA by a variation of bases in the oligonucleotide (changes are identified by underlining):

(AA-SEQ NO 33)

| | |
|---|---|
| anti-MMP-1 dsRNA: | AAG GGA AUA AGU ACU GGG CUG |
| control: | AAG GGA AAG ACG ACU GGG CUG |

Via a database search in the databases of the National Center for Biotechnical Information (BLAST Analysis) it was ensured by comparison with the previously known sequences of the complete human genome that the sequence corresponds to no coding sequence of MMP-1 (http://www.ncbi.nlm.nih.gov/BLAST/).

Cells transfected with anti-lamin A/C dsRNA served as a further control.

In the meantime the complete medium was removed from the fibroblasts and replaced by 200 μl of medium (DMEM+10% FCS) without antibiotics or serum. Then the dsRNA to which Oligofektamin (Oligofektamin reagent from Invitrogen) was added was introduced into the cells and the formulations incubated for 24 hours. An incubation period of 24 hours proved essential for an adequate transfection. After the transfection of the cells, MMP-1 expression was induced by a UV stimulus of 4 J/cm². To this end, the cells were irradiated in calcium-containing buffer (Dulbecco's phosphate-buffered saline with calcium and magnesium; Cat. No. H15-001; PAA Laboratories, Linz) and then incubated for another 48 hours. In the case of the transfected fibroblasts somewhat reduced growth was observed in comparison with untreated cells. Due to the extended incubation period however, the transfected fibroblasts otherwise exhibited no major morphological changes in comparison with untreated cells.

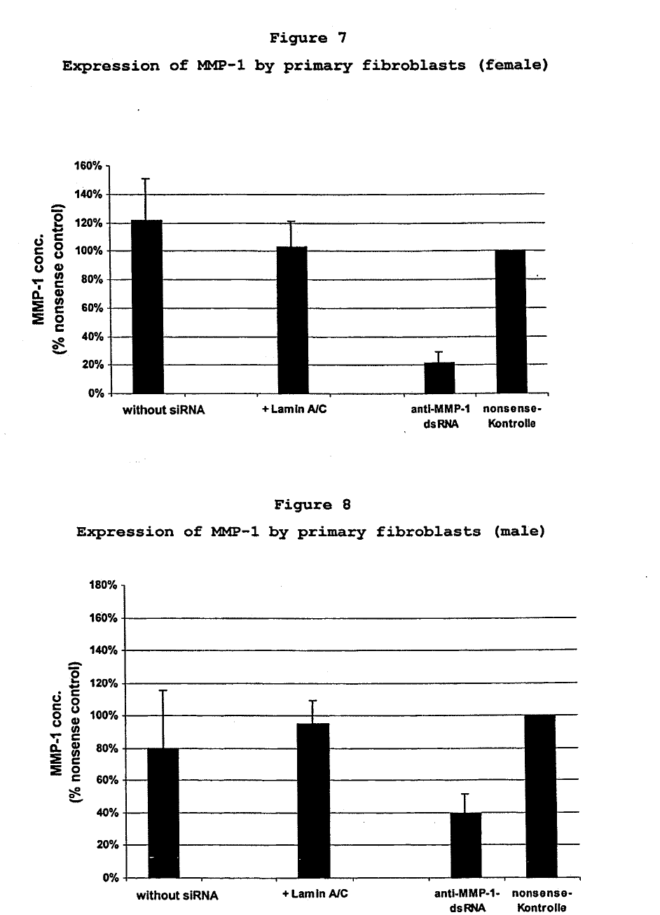

The tests were then evaluated. For this purpose the MMP-1 concentration in the cell supernatant was measured by an ELISA (MMP-1, human, Amersham Biotrak ELISA System). The ELISA is based on a two-sided “sandwich” system. The samples to be tested were incubated in a 96-well microtitre plate which was coated with an anti-MMP-1 antibody. The MMP-1 present was bound by this antibody, all the other components were removed by washing steps. Then a second polyclonal antibody was added, which was recognized from the already-bound MMP-1 band and by a peroxidase-coupled antibody. Detection took place after addition of 3,3′,5,5′-tetramethylbenzidine (TMB) and hydrogen peroxide by spectrophotometric measurement of the optical density at a wavelength of 450 nm. Repeat determinations were carried out in the case of each of the measurements. The MMP-1 concentration of the nonsense control was set to 100%. The results are shown in FIG. 7.

Transfection with the negative control dsRNA lamin A/C and the nonsense-dsRNA led to almost identical concentrations of MMP-1 in the cell supernatant. On the other hand, transfection with the anti-MMP-1 dsRNA brought about an approximately 80% reduction in the concentration of MMP-1 in the cell supernatant. The marked inhibitory effect of the anti-MMP-1 dsRNA is therefore to be observed clearly, both in the tumoral cell line HeLaS3 (Example 9) and in the cell system of the primary fibroblasts. The target sequence of the dsRNA examined here is specific to MMP-1 and cannot inhibit other MMPs.

### Example 11

### Measurement of the Inhibition of MMP1-Expression in Primary Fibroblasts (Male) by dsRNA

In order to gain an insight into donor variability, the set of tests described in Example 11 was carried out in identical manner with primary fibroblasts from a second donor (male, 42 years old, 42w). The results are shown in FIG. 8.

In the case of this donor also, the levels of the two negative controls lamin A/C and nonsense dsRNA are at the same level. Here, the transfection with the anti-MMP-1 dsRNA brought about a 60% reduction in the MMP-1 concentration. These results show that the dsRNA used also exercises a marked inhibitory effect on MMP-1 expression in different donors.

What is claimed is:

1. Double-stranded oligoribonucleotide or a physiologically compatible salt thereof which is capable of interfering with a RNA target sequence of a connective tissue decomposing enzyme.

2. Oligoribonucleotide according to claim 1, wherein the connective-tissue-decomposing enzyme comprises a collagen-decomposing endopeptidase, an elastin-decomposing endopeptidase, or a hyaluronane-decomposing endo-beta-N-acetylglycosaminidase.

3. Oligoribonucleotide according to claim 2, wherein the collagen-decomposing endopeptidase comprises matrix metalloproteinase 1, matrix metalloproteinase 8, or matrix metalloproteinase 13.

4. Oligoribonucleotide according to claim 2, wherein the elastin-decomposing endopeptidase comprises elastase 2.

5. Oligoribonucleotide according to claim 2, wherein the hyaluronane-decomposing endo-beta-N-acetylglucosaminidase comprises hyaluronidase 2 (HYAL2; U09577), SPAM1 (s67798), HYAL3 (AF036035), HYAL4 (AF009010), or HYAL5 (AF036144).

6. Oligoribonucleotide according to claim 1, wherein the oligoribonucleotide inhibits the expression of a gene of the connective tissue decomposing enzyme by at least 40%.

7. Oligoribonucleotide according to claim 1, wherein the oligoribonucleotide inhibits the expression of a gene of the connective-tissue-decomposing enzyme by at least 60%.

8. Oligoribonucleotide according to claim 1, wherein the oligoribonucleotide differs from the target sequence by a maximum of 0 to 2 base pairs relative to a length of 20 base pairs.

9. Oligoribonucleotide according to claim 1, wherein the oligoribonucleotide is 15 to 49 base pairs long.

10. Oligoribonucleotide according to claim 1, wherein the oligoribonucleotide is 19 to 25 base pairs long.

11. Oligoribonucleotide according to claim 1, wherein the oligoribonucleotide is homologous to a section of a gene of the connective-tissue decomposing enzyme, and wherein the sense strand of the gene section is flanked at a 5′ end by two adenosine radicals (A) and at a 3′ end by two thymidine radicals (T) or by one thymidine and one cytosine radical (C).

12. Oligoribonucleotide according to claim 1, wherein the oligoribonucleotide has a 3′ end which carries two desoxythymidine radicals.

13. Oligoribonucleotide according to claim 1, wherein the oligoribonucleotide is integrated into an expression vector.

14. Oligoribonucleotide according to claim 1, wherein one or more phosphate groups are replaced by phosphothioate, methylphosphonate, or phosphoramidate groups.

15. Oligoribonucleotide according to claim 1, wherein one or more ribose radicals are replaced by amino acid radicals or morpholine radicals.

16. Oligoribonucleotide according to claim 1, wherein one or more ribose radicals are modified by fluorine, alkyl, or O-alkyl radicals.

17. Oligoribonucleotide according to claim 1, wherein the oligoribonucleotide contains one or more alpha-nucleosides.

18. Pharmaceutical or cosmetic composition containing one or more oligoribonucleotides according to claim 1, or a physiologically compatible salt thereof.

19. Composition according to claim 18, wherein the composition is in a form for topical application.

20. Composition according to claim 19, wherein the composition contains several oligoribonucleotides which inhibit the expression of several different collagen-decomposing enzymes, elastases, or hyaluronidases.

21. Composition according to claim 19, wherein the composition contains several oligoribonucleotides which target different sequence regions of one and the same gene of a collagen-decomposing enzyme, an elastase, or a hyaluronidase.

22. Composition according to claim 18, wherein the composition contains 0.00001 to 10 wt.-% oligonucleotide.

23. Composition according to claim 18, wherein the composition contains 1 to 5 different oligoribonucleotides.

24. Composition according to claim 18, wherein the composition contains exclusively oligoribonucleotides which inhibit the expression of a connective tissue decomposing enzyme.

25. Composition according to claim 18, wherein the composition contains oligoribonucleotides which inhibit the expression of a hyaluronidase.

26. Composition according to claim 18, wherein the composition is present in the form of a solution, cream, ointment, lotion, hydrodispersion, lipodispersion, emulsion, Pickering emulsion, a gel, a stick, or as an aerosol.

27. A method of treatment comprising applying an oligoribonucleotide according to claim 1, or a physiologically compatible salt thereof, for care of the skin or cosmetic or therapeutic treatment of degenerative skin conditions.

28. A method of treatment comprising applying an oligoribonucleotide according to claim 1, or a physiologically compatible salt thereof, for the treatment of skin changes or skin damage which are caused by UV radiation in the connective tissue, dryness, roughness and slackness of the skin, wrinkling, reduced rehydration by sebaceous glands, and an increased susceptibility to mechanical stress (tendency to crack), for the treatment of photodermatoses, the symptoms of senile xerosis, photoaging and degenerative phenomena which are associated with a decomposition of the connective tissue of the skin.


SEQUENCE LISTING (63 sequences)

SEQ ID NO: 1 - DNA, 1970 bp, Homo sapiens
atattggagcagcaagaggctgggaagccatcacttaccttgcactgagaaagaagacaaaggccagtatgcacagctttcctccactgctgctgctgctgttctggggtgtggtgtctcacagcttcccagcgactctagaaacacaagagcaagatgtggacttagtccagaaatacctggaaaaatactacaacctgaagaatgatgggaggcaagttgaaaagcggagaaatagtggcccagtggttgaaaaattgaagcaaatgcaggaattctttgggctgaaagtgactgggaaaccagatgctgaaaccctgaaggtgatgaagcagcccagatgtggagtgcctgatgtggctcagtttgtcctcactgagggaaaccctcgctgggagcaaacacatctgaggtacaggattgaaaattacacgccagatttgccaagagcagatgtggaccatgccattgagaaagccttccaactctggagtaatgtcacacctctgacattcaccaaggtctctgagggtcaagcagacatcatgatatcttttgtcaggggagatcatcgggacaactctccttttgatggacctggaggaaatcttgctcatgcttttcaaccaggcccaggtattggaggggatgctcattttgatgaagatgaaaggtggaccaacaatttcagagagtacaacttacatcgtgttgcggctcatgaactcggccattctcttggactctcccattctactgatatcggggctttgatgtaccctagctacaccttcagtggtgatgttcagctagctcaggatgacattgatggcatccaagccatatatggacgttcccaaaatcctgtccagcccatcggcccacaaaccccaaaagcgtgtgacagtaagctaacctttgatgctataactacgattcggggagaagtgatgttctttaaagacagattctacatgcgcacaaatcccttctacccggaagttgagctcaatttcatttctgttttctggccacaactgccaaatgggcttgaagctgcttacgaatttgccgacagagatgaagtccggtttttcaaagggaataagtactgggctgttcagggacagaatgtgctacacggataccccaaggacatctacagctcctttggcttccctagaactgtgaagcatatcgatgctgctctttctgaggaaaacactggaaaaacctacttctttgttgctaacaaatactggaggtatgatgaatataaacgatctatggatccaagttatcccaaaatgatagcacatgactttcctggaattggccacaaagttgatgcagttttcatgaaagatggatttttctatttctttcatggaacaagacaatacaaatttgatcctaaaacgaagagaattttgactctccagaaagctaatagctggttcaactgcaggaaaaattgaacattactaatttgaatggaaaacacatggtgtgagtccaaagaaggtgttttcctgaagaactgtctattttctcagtcatttttaacctctagagtcactgatacacagaatataatcttatttatacctcagtttgcatatttttttactatttagaatgtagccctttttgtactgatataatttagttccacaaatggtgggtacaaaaagtcaagtttgtggcttatggattcatataggccagagttgcaaagatcttttccagagtatgcaactctgacgttgatcccagagagcagcttcagtgacaaacatatcctttcaagacagaaagagacaggagacatgagtctttgccggaggaaaagcagctcaagaacacatgtgcagtcactggtgtcaccctagataggcaagggataactcttctaacacaaaataagtgttttatgtttggaataaagtcaaccttgtttctactgtttt

SEQ ID NO: 2 - DNA, 23 bp, Homo sapiens
aagaggctgggaagccatcactt

SEQ ID NO: 3 - DNA, 21 bp, Artificial Sequence
  Positions 1 to 19 ribonucleotides; positions 20 and 21 2′-deoxythymidine
gaggcugggaagccaucactt

SEQ ID NO: 4 - DNA, 21 bp, Artificial Sequence
  Positions 1 to 19 ribonucleotides; positions 20 and 21 2′-deoxythymidine
gugauggcuucccagccuctt

SEQ ID NO: 5 - DNA, 23 bp, Homo sapiens
aaaggccagtatgcacagctttc

SEQ ID NO: 6 - DNA, 21 bp, Artificial Sequence
  Description of Artificial Sequence sense RNA
aggccaguaugcacagcuutt

SEQ ID NO: 7 - DNA, 21 bp, Artificial Sequence
  Description of Artificial Sequence antisense RNA
aagcugugcauacuggccutt

SEQ ID NO: 8 - DNA, 23 bp, Homo sapiens
aagagcaagatgtggacttagtc

SEQ ID NO: 9 - DNA, 21 bp, Artificial Sequence
  Positions 1 to 19 ribonucleotides; positions 20 and 21 2′-deoxythymidine
gagcaagauguggacuuagtt

SEQ ID NO: 10 - DNA, 21 bp, Artificial Sequence
  Description of Artificial Sequence antisense RNA
cuaaguccacaucuugcuctt

SEQ ID NO: 11 - DNA, 23 bp, Homo sapiens
aaattgaagcaaatgcaggaatt

SEQ ID NO: 12 - DNA, 21 bp, Artificial Sequence
  Description of Artificial Sequence sense RNA
auugaagcaaaugcaggaatt

SEQ ID NO: 13 - DNA, 21 bp, Artificial Sequence
  Description of Artificial Sequence antisense RNA
uuccugcauuugcuucaautt

SEQ ID NO: 14 - DNA, 23 bp, Homo sapiens
aattgaagcaaatgcaggaattc

SEQ ID NO: 15 - DNA, 21 bp, Artificial Sequence
  Description of Artificial Sequence sense RNA
uugaagcaaaugcaggaautt

SEQ ID NO: 16 - DNA, 21 bp, Artificial Sequence
  Description of Artificial Sequence antisense RNA
auuccugcauuugcuucaatt

SEQ ID NO: 17 - DNA, 23 bp, Homo sapiens
aaacacatctgaggtacaggatt

SEQ ID NO: 18 - DNA, 21 bp, Artificial Sequence
  Description of Artificial Sequence sense RNA
acacaucugagguacaggatt

SEQ ID NO: 19 - DNA, 21 bp, Artificial Sequence
  Description of Artificial Sequence antisense RNA
uccuguaccucagaugugutt

SEQ ID NO: 20 - DNA, 23 bp, Homo sapiens
aagcagacatcatgatatctttt

SEQ ID NO: 21 - DNA, 21 bp, Artificial Sequence
  Description of Artificial Sequence sense RNA
gcagacaucaugauaucuutt

SEQ ID NO: 22 - DNA, 21 bp, Artificial Sequence
  Description of Artificial Sequence antisense RNA
aagauaucaugaugucugctt

SEQ ID NO: 23 - DNA, 23 bp, Homo sapiens
aacaatttcagagagtacaactt

SEQ ID NO: 24 - DNA, 21 bp, Artificial Sequence
  Description of Artificial Sequence sense RNA
caauuucagagaguacaactt

SEQ ID NO: 25 - DNA, 21 bp, Artificial Sequence
  Description of Artificial Sequence antisense RNA
guuguacucucugaaauugtt

SEQ ID NO: 26 - DNA, 23 bp, Homo sapiens
aactcggccattctcttggactc

SEQ ID NO: 27 - DNA, 21 bp, Artificial Sequence
  Description of Artificial Sequence sense RNA
cucggccauucucuuggactt

SEQ ID NO: 28 - DNA, 21 bp, Artificial Sequence
  Description of Artificial Sequence antisense RNA
guccaagagaauggccgagtt

SEQ ID NO: 29 - DNA, 23 bp, Homo sapiens
aatttgccgacagagatgaagtc

SEQ ID NO: 30 - DNA, 21 bp, Artificial Sequence
  Description of Artificial Sequence sense RNA
uuugccgacagagaugaagtt

SEQ ID NO: 31 - DNA, 21 bp, Artificial Sequence
  Description of Artificial Sequence antisense RNA
cuucuacucugucggcaaatt

SEQ ID NO: 32 - DNA, 23 bp, Homo sapiens
aagggaataagtactgggctgtt

SEQ ID NO: 33 - DNA, 21 bp, Artificial Sequence
  Description of Artificial Sequence sense RNA
gggaauaaguacugggcugtt

SEQ ID NO: 34 - DNA, 21 bp, Artificial Sequence
  Description of Artificial Sequence antisense RNA
cagcccaguacuuauuccctt

SEQ ID NO: 35 - DNA, 23 bp, Homo sapiens
aacactggaaaaacctacttctt

SEQ ID NO: 36 - DNA, 21 bp, Artificial Sequence
  Description of Artificial Sequence sene RNA
cacuggaaaaaccuacuuctt

SEQ ID NO: 37 - DNA, 21 bp, Artificial Sequence
  Description of Artificial Sequence antisense RNA
gaaguagguuuuuccagugtt

SEQ ID NO: 38 - DNA, 23 bp, Homo sapiens
aaaacactggaaaaacctacttc

SEQ ID NO: 39 - DNA, 21 bp, Artificial Sequence
  Description of Artificial Sequence sense RNA
aacacuggaaaaaccuacutt

SEQ ID NO: 40 - DNA, 21 bp, Artificial Sequence
  Description of Artificial Sequence antisense RNA
aguagguuuuuccaguguutt

SEQ ID NO: 41 - DNA, 23 bp, Homo sapiens
aacaagacaatacaaatttgatc

SEQ ID NO: 42 - DNA, 21 bp, Artificial Sequence
  Description of Artificial Sequence sense RNA
caagacaauacaaauuugatt

SEQ ID NO: 43 - DNA, 21 bp, Artificial Sequence
  Description of Artificial Sequence antisense RNA
ucaaauuuguauugucuugtt

SEQ ID NO: 44 - DNA, 23 bp, Homo sapiens
aaacgaagagaattttgactctc

SEQ ID NO: 45 - DNA, 21 bp, Artificial Sequence
  Description of Artificial Sequence sense RNA
acgaagagaauuuugacuctt

SEQ ID NO: 46 - DNA, 21 bp, Artificial Sequence
  Description of Artificial Sequence antisense RNA
gagucaaaauucucuucgutt

SEQ ID NO: 47 - DNA, 23 bp, Homo sapiens
aactgcaggaaaaattgaacatt

SEQ ID NO: 48 - DNA, 21 bp, Artificial Sequence
  Description of Artificial Sequence sense RNA
cugcaggaaaaauugaacatt

SEQ ID NO: 49 - DNA, 21 bp, Artificial Sequence
  Description of Artificial Sequence antisense RNA
uguucaauuuuuccugcagtt

SEQ ID NO: 50 - DNA, 23 bp, Homo sapiens
aagaactgtctattttctcagtc

SEQ ID NO: 51 - DNA, 21 bp, Artificial Sequence
  Description of Artificial Sequence sense RNA
gaacugucuauuuucucagtt

SEQ ID NO: 52 - DNA, 21 bp, Artificial Sequence
  Description of Artificial Sequence antisense RNA
cugagaaaauagacaguuctt

SEQ ID NO: 53 - DNA, 23 bp, Homo sapiens
aagagacaggagacatgagtctt

SEQ ID NO: 54 - DNA, 21 bp, Artificial Sequence
  Description of Artificial Sequence sense RNA
gagacaggagacaugaguctt

SEQ ID NO: 55 - DNA, 21 bp, Artificial Sequence
  Description of Artificial Sequence antisense RNA
gacucaugucuccugucuctt

SEQ ID NO: 56 - DNA, 23 bp, Homo sapiens
aagtcaaccttgtttctactgtt

SEQ ID NO: 57 - DNA, 21 bp, Artificial Sequence
  Description of Artificial Sequence sense RNA
gucaaccuuguuucuacugtt

SEQ ID NO: 58 - DNA, 21 bp, Artificial Sequence
  Description of Artificial Sequence antisense RNA
caguagaaacaagguugactt

SEQ ID NO: 59 - DNA, 920 bp, Homo sapiens
gcacggaggggcagagaccccggagccccagccccaccatgaccctcggccgccgactcgcgtgtcttttcctcgcctgtgtcctgccggccttgctgctggggggcaccgcgctggcctcggagattgtggggggccggcgagcgcggccccacgcgtggcccttcatggtgtccctgcagctgcgcggaggccacttctgcggcgccaccctgattgcgcccaacttcgtcatgtcggccgcgcactgcgtggcgaatgtaaacgtccgcgcggtgcgggtggtcctgggagcccataacctctcgcggcgggagcccacccggcaggtgttcgccgtgcagcgcatcttcgaaaacggctacgaccccgtaaacttgctcaacgacatcgtgattctccagctcaacgggtcggccaccatcaacgccaacgtgcaggtggcccagctgccggctcagggacgccgcctgggcaacggggtgcagtgcctggccatgggctggggccttctgggcaggaaccgtgggatcgccagcgtcctgcaggagctcaacgtgacggtggtgacgtccctctgccgtcgcagcaacgtctgcactctcgtgaggggccggcaggccggcgtctgtttcggggactccggcagccccttggtctgcaacgggctaatccacggaattgcctccttcgtccggggaggctgcgcctcagggctctaccccgatgcctttgccccggtggcacagtttgtaaactggatcgactctatcatccaacgctccgaggacaacccctgtccccacccccgggacccggacccggccagcaggacccactgagaagggctgcccgggtcacctcagctgcccacacccacactctccagcatctggcacaataaacattctctgttttgt

SEQ ID NO: 60 - DNA, 23 bp, Homo sapiens
aacggctacgaccccgtaaactt

SEQ ID NO: 61 - DNA, 1848 bp, Homo sapiens
ggctcaccccaggtaaggagggaggccaccgacctactgggccgacggactcccacacagttcctgagctggtgccaggcaggtgacacctcctgcagcccccagcatgcgggcaggcccaggccccaccgttacattggccctggtgctggcggtgtcatgggccatggagctcaagcccacagcaccacccatcttcactggccggccctttgtggtagcgtgggacgtgcccacacaggactgtggcccacgcctcaaggtgccactggacctgaatgcctttgatgtgcaggcctcacctaatgagggttttgtgaaccagaatattaccatcttctaccgcgaccgtctaggcctgtatccacgcttcgattctgccggaaggtctgtgcatggtggtgtgccacagaatgtcagcctttgggcacaccggaagatgctgcagaaacgtgtggagcactacattcggacacaggagtctgcggggctggcggtcatcgactgggaggactggcgacctgtgtgggtgcgcaactggcaggacaaagatgtgtatcgccggttatcacgccagctagtggccagtcgtcaccctgactggcctccagaccgcatagtcaaacaggcacaatatgagtttgagttcgcagcacagcagttcatgctggagacactgcgttatgtcaaggcagtgcggccccggcacctctggggcttctacctctttcctgactgctacaatcatgattatgtgcagaactgggagagctacacaggccgctgccctgatgttgaggtggcccgcaatgaccagctggcctggctgtgggctgagagcacggccctcttcccgtctgtctacctggacgagacacttgcttcctcccgccatggccgcaactttgtgagcttccgtgttcaggaggcccttcgtgtggctcgcacccaccatgccaaccatgcactcccagtctacgtcttcacacgacccacctacagccgcaggctcacggggcttagtgagatggacctcatctctaccattggcgagagtgcggccctgggcgcagctggtgtcatcctctggggtgacgcggggtacaccacaagcacggagacctgccagtacctcaaagattacctgacacggctgctggtcccctacgtggtcaatgtgtcctgggccacccaatattgcagccgggcccagtgccatggccatgggcgctgtgtgcgccgcaaccccagtgccagtaccttcctgcatctcagcaccaacagtttccgcctagtgcctggccatgcacctggtgaaccccagctgcgacctgtgggggagctcagttgggccgacattgaccacctgcagacacacttccgctgccagtgctacttgggctggagtggtgagcaatgccagtgggaccataggcaggcagctggaggtgccagcgaggcctgggctgggtcccacctcaccagtctgctggctctggcagccctggcctttacctggaccttgtaggggtctcctgcctagctgcctagcaagctggcctctaccacaagggctctcttaggcatgtaggaccctgcagggggtggacaaactggagtctggagtgggcagagcccccaggaagcccaggagggcatccataccagctcgcacccccctgttctaagggggaggggaagtccctgggaggccccttctctccctgccagaggggaaggagggtacagctgggctggggaggacctgaccctactcccttgccctagatagtttattattattattattttggggtctcttttgtaaattaaacataaaacaattgc

SEQ ID NO: 62 - DNA, 23 bp, Homo sapiens
aatgcctttgatgtgcaggcctc

SEQ ID NO: 63 - DNA, 23 bp, Homo sapiens
aaagatgtgtatcgccggttatc